(19) United States
(12) Patent Application Publication
(10) Pub. No.: US 2010/0197944 A1
(43) Pub. Date: Aug. 5, 2010

(54) DOCETAXEL PROCESS AND POLYMORPHS

(75) Inventors: Raghavendracharyulu Venkata Palle, Hyderabad (IN); Sekhar Munaswamy Nariyam, Hyderabad (IN); Rajasekhar Kadaboina, Hyderabad (IN); Lankeshwararao Matti, Hyderabad (IN); Sridhar Vasam, Warangal (IN); Shravan Kumar Komati, Nalgonda (IN); Siva Reddy Makireddy, Guntur (IN)

Correspondence Address: DR. REDDY''S LABORATORIES, INC., 200 SOMERSET CORPORATE BLVD, SEVENTH FLOOR, BRIDGEWATER, NJ, 08807-2862, US

(73) Assignees: DR. REDDY'S LABORATORIES LIMITED, Hyderabad 500016, Andhra Pradesh (IN); DR. REDDY'S LABORATORIES, INC., Bridgewater, NJ (US)

(21) Appl. No.: 12/667,608

(22) PCT Filed: Jul. 3, 2008
(86) PCT No.: PCT/US2008/069192

§ 371 Date: Jan. 4, 2010

Related U.S. Application Data
(60) Provisional application No. 60/986,027, filed on Nov. 7, 2007.
(60) Provisional application No. 61/037,162, filed on Mar. 17, 2008.

(30) Foreign Application Priority Data
Jul. 4, 2007 (IN) 1442/CHE/2007
Aug. 22, 2007 (IN) 1880/CHE/2007

(51) Int. Cl. C07D 305/14

(52) U.S. Cl. 549/510

(57) ABSTRACT

Processes for preparing substantially pure docetaxel, new crystalline forms of docetaxel and processes for preparation thereof, processes for preparing docetaxel trihydrate, and pharmaceutical compositions comprising docetaxel.

### INTRODUCTION

The present invention relates to a process for the preparation of substantially pure docetaxel. The present invention also relates to new crystalline forms of Docetaxel and process for their preparation. The present invention also relates to a process for preparing docetaxel trihydrate. Further, the present invention relates to pharmaceutical compositions of docetaxel that are useful in the treatment of various cancerous disorders.

Docetaxel is the adopted name for a drug compound having a chemical name (2R,3S)—N-carboxy-3-phenylisoserine,N-tert-butyl ester, 13-ester with 5β-20-epoxy-1,2α,4,7β,10β,13β-hexahydroxytax-11-en-9-one 4-acetate 2-benzoate and is represented by structural Formula I.

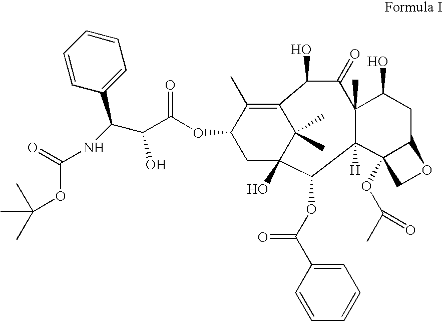

Docetaxel has activity against several kinds of cancer, including breast cancer, non-small cell lung cancer and other malignant tumours. Docetaxel is an antineoplastic agent belonging to the taxoid family and is available in products sold with the brand name TAXOTERE® in the form of a sterile, non-pyrogenic injection in single-dose vials containing 20 mg (0.5 mL) or 80 mg (2 mL) of the drug.

TAXOTERE® injection comprises a two component formulation that requires two-step dilution before infusion. The first step involves dilution with the contents of a diluent vial (13% w/w ethanol in water for injection) without significant foaming, and the second step involves further dilution with infusion fluid for parenteral administration.

A number of taxol analogs have been described by F. Guéritte-Voegelein et al., “Relationships between the Structure of Taxol Analogues and Their Antimitotic Activity,” Journal of Medicinal Chemistry, Vol. 34, pages 992-998, 1991.

Colin et al., U.S. Pat. No. 4,814,470, discloses docetaxel, its stereoisomeric forms, pharmaceutical compositions containing docetaxel and their use in the treatment of acute leukemias and solid tumours.

L Zaske et al., “Docetaxel: Solid state characterization by X-ray powder diffraction and thermogravimetry,” Journal de Physique IV France, Vol. 11, Pr10-221 to Pr10-226 (2001) discloses the solid-state characterization of anhydrous, hemihydrate and trihydrate forms of docetaxel.

Page et al., in U.S. Pat. No. 6,002,025, discloses a process for the purification of taxanes using column chromatography with a phenylalkyl resin.

International Application Publication No. WO 2007/044950 A2 discloses crystalline forms of docetaxel and processes for their preparation.

Durand et al, in U.S. Pat. No. 6,197,980, discloses 4-acetoxy-2α-benzoyloxy-5β,20-epoxy-1β,7β,10β-trihydroxy-9-oxo-11-taxen-13α-y1 (2R,3S)-3-t-butoxycarbonylamino-3-phenyl-2-hydroxypropionate trihydrate obtained by a process of centrifugal partition chromatography, comprising centrifuging impure 4-acetoxy-2α-benzoyloxy-5β,20-epoxy-1β,7β,10β-trihydroxy-9-oxo-11-taxen-13 α-yl (2R,3S)-3-t-butoxy carbonyl amino-3-phenyl-2-hydroxypropionate and at least four solvents, wherein the solvents are an aliphatic hydrocarbon, an ester, an alcohol, and water.

Authelin et al., in U.S. Pat. No. 6,022,985, disclose a process for the preparation of Docetaxel trihydrate, wherein Docetaxel is crystallized from a mixture of water and an aliphatic alcohol containing 1 to 3 carbon atoms, and then the product obtained is dried under defined conditions of temperature, pressure and humidity. Further, the patent discloses that 4-acetoxy-2α-benzoyloxy-5β,20-epoxy-1,7β,10β-trihydroxy-9-oxo-tax-11-en-13-α-yl-(2R,3S)-3-tert-butoxycarbonylamino-2-hydroxy-3-phenylpropionate trihydrate has a substantially greater stability than that of the anhydrous product.

Sharma et al., in U.S. Pat. No. 6,838,569, disclose a process for converting paclitaxel or docetaxel to the respective trihydrate in a mixture of alkane and chlorinated alkane to provide a crude product of 65-75% purity and the crude product subsequently dissolved in an alkyl ketone, followed by addition of an alkane to provide a product of enhanced chromatographic purity, which in an aliphatic nitrile, followed by addition of water provides taxane trihydrate.

International Application Publication No. WO 2005/061474 A1 discloses a process for the preparation of amorphous, anhydrous, and trihydrate forms of docetaxel. The process for docetaxel trihydrate involves solubilizing docetaxel in a solvent which is chemically inert. This solvent may be a linear or branched alcohol (containing between 1 and 8 carbons), an organic acid, an aliphatic or cyclic ether, a polar, aprotic solvent, a halogenated solvent, an aromatic solvent, a polyethoxylated sorbitol, lecithin or castor oil, or another solvent of adequate polarity to effect the complete solubilization of docetaxel and is capable of solubilizing, or is miscible with, at least 3 molar equivalents of water. The solution so obtained is admixed with an amount of distilled water between 3 and 200,000 molar equivalents relative to docetaxel. Crystallization is induced and the docetaxel trihydrate is isolated by means of conventional processes such as filtration, decantation or centrifugation.

Li Jinliang et al., in U.S. Patent Application Publication No. 2006/0217436 A1, discloses a process for preparing docetaxel trihydrate, which involves repeated dissolution and removal of the solvent by concentration of docetaxel in acetone. The final precipitation of the product is by addition of water to the solution of the compound in acetone.

The above mentioned documents disclose diverse processes for the preparation of docetaxel trihydrate, but due to one more reasons they are not particularly convenient and amenable to commercial scale-up for preparing docetaxel trihydrate. Thus, there is an unmet need for a simple, cost-effective process for the preparation of docetaxel trihydrate, which overcomes the drawbacks of various prior disclosed processes, e.g., multiple solvent combinations as well as multiple steps, which make the processes neither cost effective nor amenable to scale up for industrial scale production.

Further, the existence of different polymorphs is a property of some compounds. A single compound, or a salt complex, may give rise to a variety of solids having distinct physical properties. This variation frequently results in bioavailability, stability, and other differences between production lots of formulated pharmaceutical products. Owing to the reason that-polymorphic forms can vary in their chemical and physical properties, regulatory authorities often require that efforts be made to identify all polymorphic forms, e.g., crystalline, amorphous, solvated forms, etc. of the drug substances. However, the existence, and possible numbers, of polymorphic forms for a given compound cannot be predicted. In addition, there are no “standard” procedures that can be used to prepare polymorphic forms of a substance.

New forms of pharmaceutically useful compounds provide an opportunity to improve the performance characteristics of such product. Further, discovery of additional polymorphic forms may help in the identification of the polymorphic content of a batch of an active pharmaceutical ingredient. Therefore, there is a need for preparing new solid forms of a drug substance and processes for preparation thereof.

According to the present invention there are provided new crystalline forms of docetaxel, and processes for their preparation, a process for preparing docetaxel trihydrate which is simple, eco-friendly, cost-effective, robust and well-suited for use on an industrial scale, and pharmaceutical compositions comprising docetaxel.

### SUMMARY

The present invention provides processes for the preparation of substantially pure docetaxel, new crystalline forms of docetaxel and processes for their preparation.

The present application also provides processes for preparing docetaxel trihydrate and pharmaceutical compositions comprising docetaxel.

An embodiment of the present invention provides a process for the preparation of a compound having Formula A, which comprises:

a) reacting a compound of Formula B with zinc, in the presence of acetic acid, to give a compound of Formula A;

b) purifying the compound of Formula A using ethyl acetate and n-heptane; and

c) converting the compound of Formula A to docetaxel.

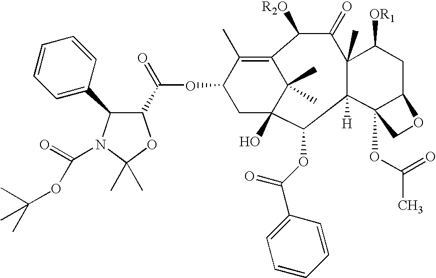

In Formula A, R₁═R₂═H. In Formula B, R₁═R₂═CCl₃—CH₂—OCO—.

An aspect of the present invention provides processes for the preparation of substantially pure docetaxel, an embodiment comprising three steps of purification:

1. Purification using acetonitrile and ether, comprising:

a) dissolving crude docetaxel (such as having a purity about 82% or less) in acetonitrile;

b) crystallizing docetaxel by adding an ether;

c) separating the crystals and washing with an ether; and

d) optionally, recrystallizing the docetaxel obtained; to recover docetaxel having enhanced purity (such as greater than about 90%).

2. Column purification comprising:

a) dissolving crude docetaxel (such as having a purity about 90% or higher) in dichloromethane to get a clear solution;

b) eluting the docetaxel-dichloromethane solution through a silica column using eluents comprising ethyl acetate and n-heptane;

c) mixing eluent fractions obtained from the silica column;

d) reducing the volume of solvent of the eluent by distillation;

e) cooling the concentrate of d) to about 25-30° C.;

f) filtering the formed solid material and washing with n-heptane; and

g) drying the wet material under vacuum; to recover docetaxel having enhanced purity (such as greater than about 95%).

3. Purification using acetone and ether, comprising:

a) dissolving crude docetaxel (such as having a purity of about 95% or higher) in acetone;

b) crystallizing docetaxel from the solution of a) by adding an ether;

c) filtering formed crystals and washing with ether; and

d) optionally, recrystallizing the docetaxel obtained; to recover substantially pure docetaxel (such as having a purity about 99.5% or higher).

Yet another aspect of the present invention provides new crystalline forms of docetaxel.

In embodiments, the present invention provides crystalline forms of docetaxel hereinafter referred to as “Form X” and “Form XI.”

In an embodiment, the present invention provides docetaxel crystalline Form X characterized by an X-ray powder diffraction (XRPD) pattern with characteristic peaks at diffraction angles 2-theta of about 5.3, 8.9, 10.0, 10.6, 11.2, 12.2, 13.7, 14.1, 15.8, 20.4, 21.2, 21.6, and 21.9, ±0.2 degrees.

In an embodiment, the present invention provides docetaxel crystalline Form X characterized by a differential scanning calorimetry (DSC) thermogram curve having endotherm peaks at about 106° C. and 175° C.

In an embodiment, the present invention provides docetaxel crystalline Form X characterized by a thermogravimetric analysis (TGA) curve corresponding to a weight loss of about 2.6%.

In an embodiment, the present invention provides crystalline Form X of docetaxel characterized by an infrared (IR) absorption spectrum in a potassium bromide (KBr) pellet substantially as represented by the spectrum of FIG. 4.

In an embodiment, the present invention provides docetaxel crystalline Form XI characterized by an XRPD pattern with characteristic peaks at diffraction angles 2-theta of about 4.4, 4.5, 7.0, 8.0, 8.7, 9.1, 11.0, 11.4, 12.3, 12.5, 13.5, 14.1, 15.4, 16.5, 16.9, 17.4, 18.4, 19.5, and 20.4, ±0.2 degrees.

In an embodiment, the present invention provides docetaxel crystalline Form XI characterized by a DSC thermogram curve with an endotherm having an onset at about 66° C. and an endset at about 161° C.

In an embodiment, the present invention provides docetaxel crystalline Form XI characterized by a TGA curve corresponding to a weight loss of about 1.9%.

In another aspect, the present invention provides crystalline Form XI characterized by an IR absorption spectrum in a potassium bromide (KBr) pellet substantially as represented by the spectrum of FIG. 8.

An aspect of the present invention provides processes for the preparation of the crystalline forms X and XI of docetaxel.

In an embodiment, the present invention provides a process for the preparation of crystalline Form X of docetaxel, comprising:

a) providing a solution of docetaxel in ethyl acetate;

b) crystallizing a solid from the solution;

c) isolating the obtained solid; and

d) drying.

In an embodiment, the present invention provides a process for the preparation of crystalline Form XI of docetaxel, comprising:

a) providing a solution of docetaxel in acetone;

b) precipitating a solid by adding an ether;

c) isolating the obtained solid; and

d) drying.

An aspect of the present invention provides processes for preparing docetaxel trihydrate, an embodiment comprising:

a) providing a mixture of docetaxel and water;

b) maintaining the mixture at 25-30° C. with stirring;

c) isolating the solid obtained; and

d) drying.

An aspect of the present invention provides pharmaceutical compositions comprising docetaxel and processes for their preparation. In an aspect, the present invention relates to pharmaceutical compositions of docetaxel for parenteral administration, upon dilution with aqueous fluids.

In an aspect, the present invention provides pharmaceutical compositions comprising docetaxel, comprising at least a two component system, wherein:

a) component 1 comprises a therapeutically effective amount of docetaxel, polyethylene glycol 660 12-hydroxystearate, and an alcohol; and

b) component 2 comprises polyethylene glycol 660 12-hydroxystearate, and an alcohol.

In an aspect, the present invention provides a kit suitable for reconstitution using an aqueous fluid for parenteral administration, wherein said kit comprises a two-component pharmaceutical composition, wherein:

a) component 1 comprises a therapeutically effective amount of docetaxel, polyethylene glycol 660 12-hydroxystearate, and an alcohol; and

b) component 2 comprises polyethylene glycol 660 12-hydroxystearate, and an alcohol.

In an embodiment, the contents of the two components of a kit of the present invention can be mixed in defined proportions to obtain a “pre-mix”, which is further diluted with aqueous dilution fluid for parenteral administration.

In an embodiment, the docetaxel compositions of the present invention, upon dilution with an aqueous fluid, are administered parenterally to a mammal in need of docetaxel therapy, wherein such therapy exhibits reduced toxic manifestations that are associated with polysorbates.

### BRIEF DESCRIPTION OF THE DRAWINGS

FIG. 1 is an X-ray powder diffraction (XRPD) pattern of docetaxel Form X prepared according to Example 4.

FIG. 2 is a differential scanning calorimetry (DSC) thermogram curve of docetaxel Form X prepared according to Example 4.

FIG. 3 is a thermogravimetric analysis (TGA) curve of docetaxel Form X prepared according to Example 4.

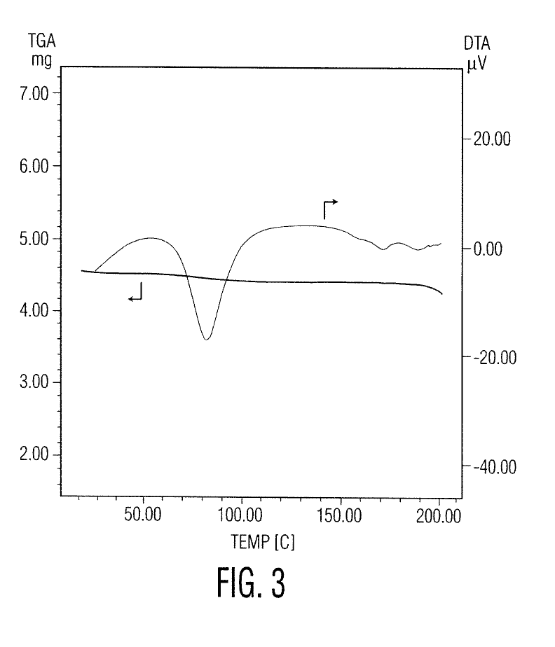

FIG. 4 is an infrared (IR) absorption spectrum of docetaxel Form X prepared according to Example 4.

FIG. 5 is an XRPD pattern of docetaxel Form XI prepared according to Example 7.

FIG. 6 is a DSC thermogram of docetaxel Form XI prepared according to Example 7.

FIG. 7 is a TGA curve of docetaxel Form XI prepared according to Example 7.

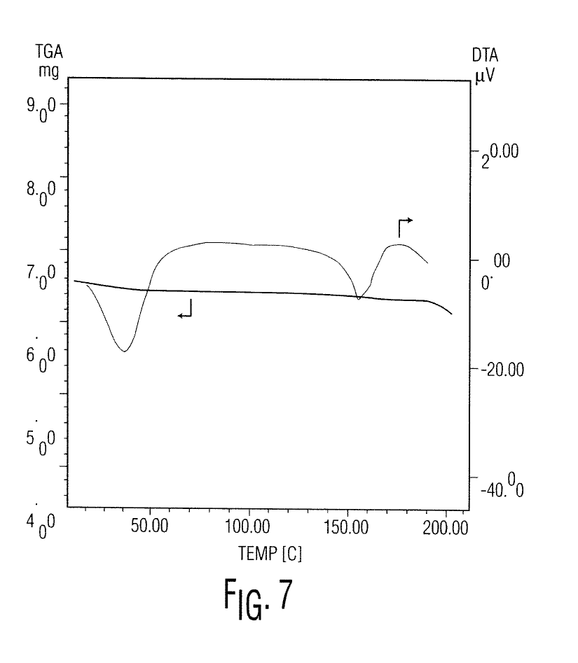

FIG. 8 is an IR absorption spectrum of docetaxel Form XI prepared according to Example 7.

FIG. 9 is an XRPD pattern of docetaxel trihydrate prepared according to Example 9.

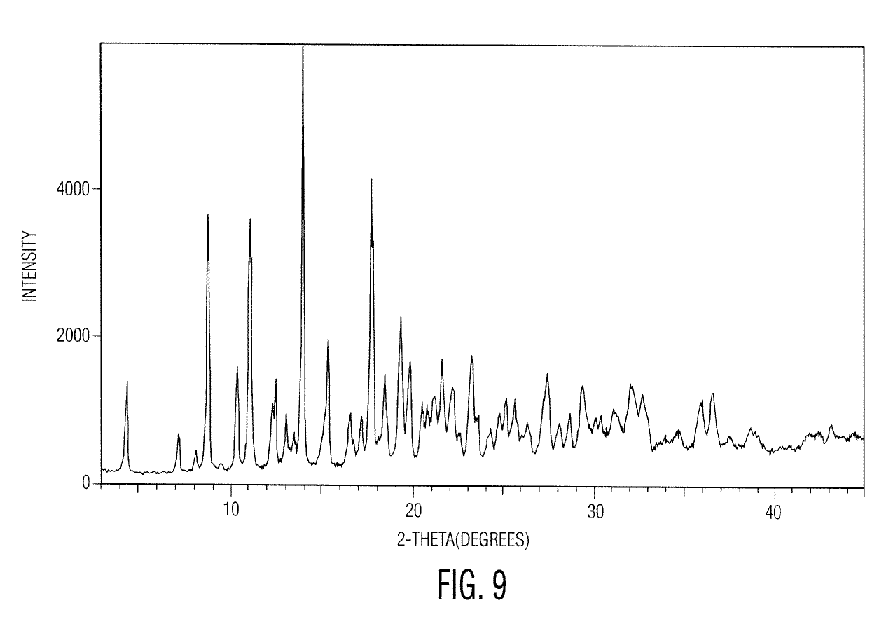

FIG. 10 is a DSC thermogram of docetaxel trihydrate prepared according to Example 9.

FIG. 11 is a TGA curve of docetaxel trihydrate prepared according to Example 9.

FIG. 12 is an IR absorption spectrum of docetaxel trihydrate prepared according to Example 9.

FIG. 13 is an XRPD pattern of docetaxel prepared according to Example 1, Step B.

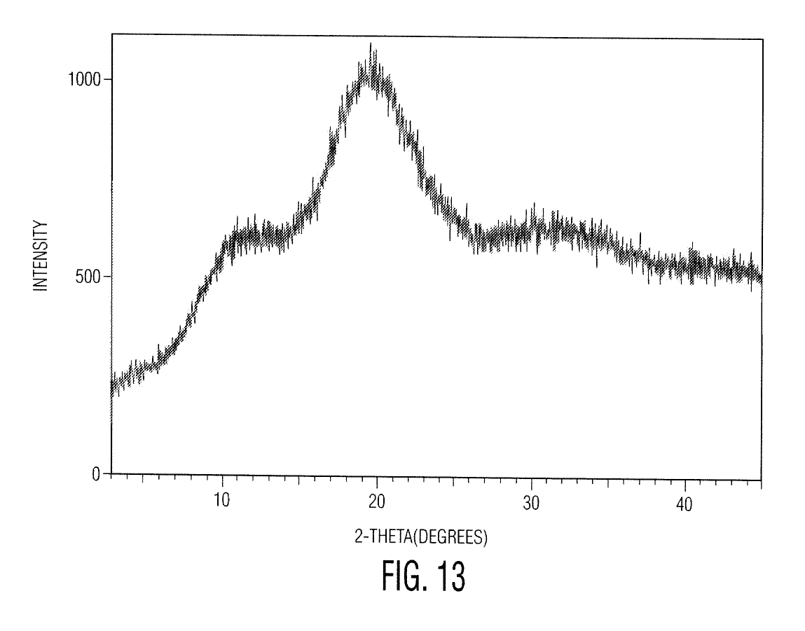

FIG. 14 is an XRPD pattern of docetaxel prepared according to Example 1, Step C, Purification 1.

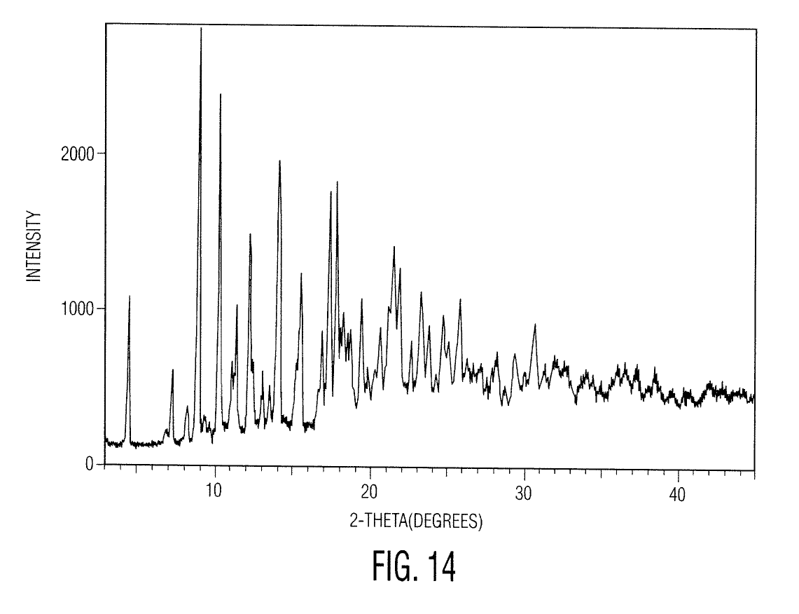

FIG. 15 is an XRPD pattern of docetaxel prepared according to Example 1, Step C, Purification 2.

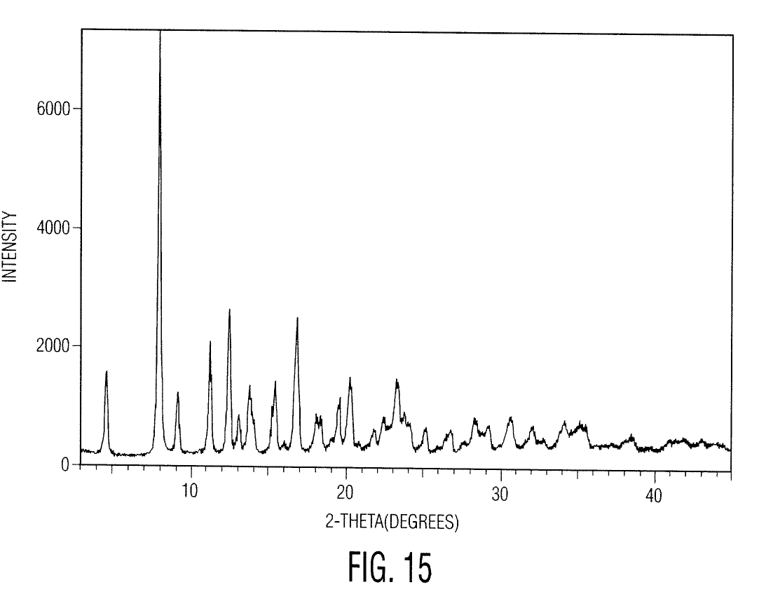

FIG. 16 is an XRPD pattern of docetaxel prepared according to Example 1, Step D.

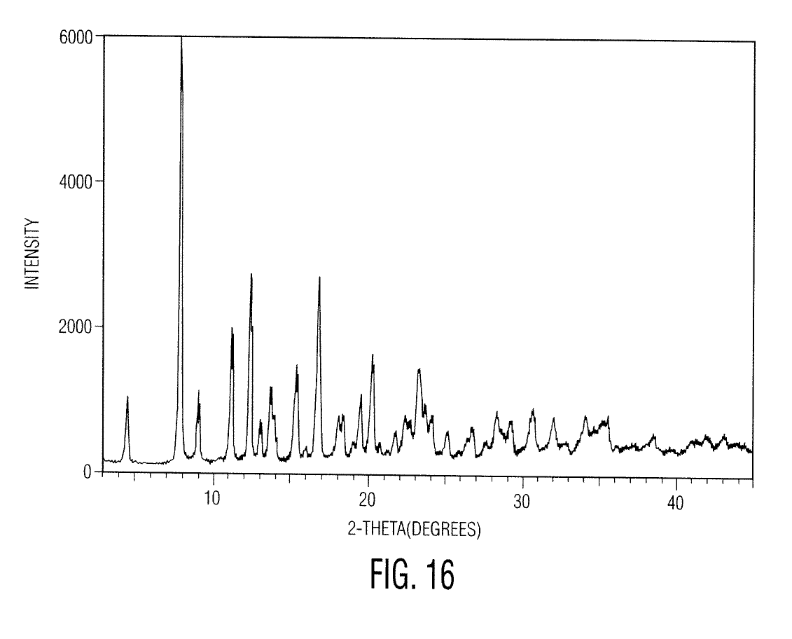

### DETAILED DESCRIPTION

The present invention provides processes for the preparation of substantially pure docetaxel, new crystalline forms of docetaxel, and processes for the preparation thereof.

The present application also provides processes for preparing docetaxel trihydrate and pharmaceutical compositions comprising docetaxel.

X-ray analysis information presented herein was obtained using copper K-α radiation. Purities and other percentages are expressed on a weight basis, unless the context clearly indicates otherwise.

An aspect of the present invention provides processes for the preparation of docetaxel, an embodiment comprising:

a) reacting a compound of Formula-B with zinc and acetic acid in the presence of methanol to give a compound of Formula A;

b) purifying the compound of Formula A using ethyl acetate and n-heptane; and

c) converting the compound of Formula A to docetaxel.

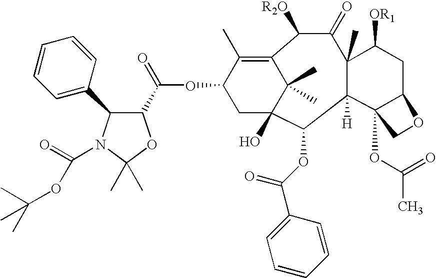

In Formula A, R₁═R₂═H. In Formula B, R₁═R₂═CCl₃—CH₂—OCO—.

All of the steps for this process are separately described below.

Step a) involves reacting the compound of Formula B with zinc and acetic acid in the presence of methanol to give a compound of Formula A.

The amount of zinc used in step a) can range from about 1 to about 20 molar equivalents, or about 8 molar equivalents, per molar equivalent of Formula B. The zinc can be used in any form, such as a powder, turnings, granules, etc.

The concentration of acetic acid used in step a) is from about 95 to about 100%. The quantity of acetic acid can range from about 1 to about 15 L, per kg of the compound of Formula B.

Suitable alcohols that can be used include but are not limited to methanol, ethanol, isopropyl alcohol, and the like.

After completion of the reaction, the reaction mixture can be filtered to remove zinc and then the solid is isolated by combining the solution with an anti-solvent such as water to give a crude compound of Formula A

Step b) involves purifying the compound of Formula A using ethyl acetate and n-heptane.

The obtained compound of Formula A from a) is purified once or repeatedly, to get the desired purity of the compound of Formula A, which is substantially free of its process-related impurities of Formula C, Formula D, Formula E, and Formula F, the structures of which are described in Table I.

TABLE 1

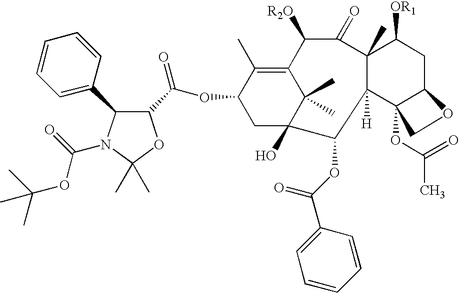

| Compound | R₁ | R₂ |
|---|---|---|
| Formula A | —H | —H |
| Formula B | 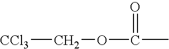 | 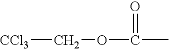 |
| Formula C | 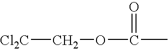 | —H |
| Formula D | —H | 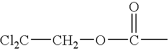 |

| | |
|---|---|
| Formula E | 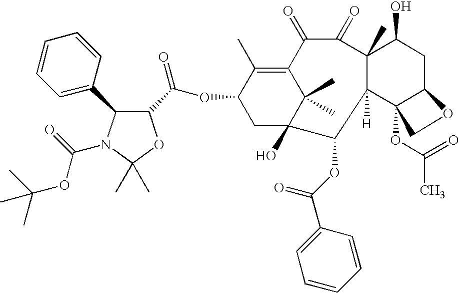 |
| Formula F | 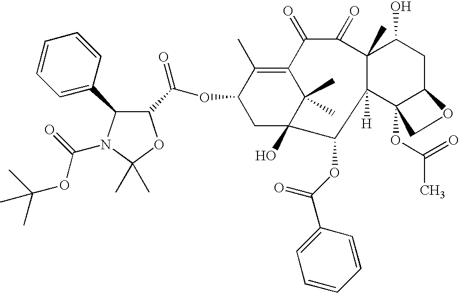 |

Purification of the compound of Formula A can carried out by dissolving a crude compound of Formula A (such as having a purity less than about 80%) in a suitable solvent and then precipitating by adding an anti-solvent. Suitable solvents that can be used for the dissolution include, but are not limited to, ethyl acetate, isobutyl acetate, n-butyl acetate, n-propyl acetate, isopropyl acetate, and the like. Anti-solvents that can be used for the precipitation include, but are not limited to, C₄ to C₁₀ straight or branched alkanes or cycloalkanes such as n-pentane, n-hexane, n-heptane, cyclohexane, and the like, and aromatic hydrocarbons such as benzene, toluene, xylene, and the like.

In a specific embodiment, the compound of Formula A is purified by dissolving the crude compound of Formula A in ethyl acetate, and precipitating the compound by adding n-heptane.

The compound of Formula A obtained by the processes of the present invention is substantially free of its structural impurities. More specifically, the compound of Formula A has a purity of not less than about 90% by high performance liquid chromatography (“HPLC”).

Step c) involves converting the compound of Formula A to docetaxel.

The compound of Formula A can be further converted to docetaxel, according using a process described in International Application Publication No. WO 2007/109654 A2, which is incorporated herein by this reference.

Another aspect of the present invention provides processes for the preparation of substantially pure docetaxel, an embodiment comprising three steps of purification:

1. Purification using acetonitrile and ether, comprising:

a) dissolving crude docetaxel (such as having a purity less than about 82%) in acetonitrile;

b) crystallizing docetaxel from the solution by adding an ether; and

c) optionally, recrystallizing the docetaxel obtained; to provide docetaxel having enhanced purity (such as at least about 90%).

2. Column purification, comprising:

a) dissolving crude docetaxel (such as having a purity about 90% or higher) in dichloromethane to get a clear solution;

b) eluting the docetaxel solution through a silica column using eluents comprising ethyl acetate and n-heptane;

c) mixing eluent fractions obtained from the silica column;

d) reducing the volume of solvent of the eluent;

e) cooling the concentrate of d) to form crystals; and

f) optionally, drying to give docetaxel having enhanced purity (such as higher than about 95%).

3. Purification using acetone and ether, comprising:

a) dissolving docetaxel (such as having purity of about 95% or higher) in acetone;

b) crystallizing docetaxel from the solution of a) by adding an ether; and

c) optionally, recrystallizing the docetaxel obtained; to recover substantially pure docetaxel (such as having a purity about 99.5% or higher).

The steps for purification by this process of the present invention are separately described below.

1. Purification using acetonitrile and ether.

Docetaxel obtained from any synthesis methods can be used as a starting material.

Docetaxel (such as having a purity about 82% or less) can be purified by crystallization, comprising combining a mixture of docetaxel in acetonitrile with an anti-solvent.

The concentration of docetaxel in the solution is not critical, but the quantity of solvent employed is usually kept to a minimum, to avoid excessive product losses during the crystallization of solid. The concentration of docetaxel in the solution may generally range from about 0.01 to about 0.5 g/ml.

The mixture of docetaxel and acetonitrile can be prepared at temperatures ranging from about 25° C. to 75° C. Depending on the quantity of solvent used, docetaxel may dissolve at a temperature of about 25° C. to 35° C., or the solution may need to be heated to elevated temperatures.

Docetaxel can be precipitated from the solution by combining with an anti-solvent. Useful anti-solvents include but are not limited to: alkanes such as n-heptane, n-hexane, cyclohexane and the like; ethers such as diethyl ether, diisopropyl ether, methyl tertiary-butyl ether and the like; and combinations thereof.

Suitable temperatures for solid crystallization can range from about 0° C. to about 80° C., or from about 25° C. to about 35° C.

The obtained precipitate may be separated using conventional techniques known in the art. One skilled in the art may appreciate that there are many ways to separate a solid from the mixture, for example it can be separated by using any techniques such as filtration by gravity or by suction, centrifugation, decantation, and the like. After separation, the solid may optionally be washed with a suitable anti-solvent.

The isolated solid may optionally be further dried. Drying can be suitably carried out using equipment such as a tray dryer, vacuum oven, air oven, fluidized bed drier, spin flash dryer, flash dryer and the like. The drying may be carried out at temperatures of about 35° C. to about 70° C., optionally under reduced pressure. The drying can be carried out for any time periods necessary for obtaining a desired purity, such as about 1 to about 25 hours, or longer.

In an embodiment, the purification of docetaxel is carried out by providing a mixture of docetaxel in acetonitrile and precipitating the compound by adding diisopropyl ether.

The above crystallization process can afford docetaxel typically having purity greater than or equal to about 85%, or greater than or equal to about 90%.

2. Column Purification of step 1 purified material.

Purification of docetaxel obtained from step 1 (such as having a purity about 90% or higher) can be carried out by column chromatography using a silica gel having a particle size range such as for example 230-400 mesh, 100-200 mesh, 60-100 mesh, or 500-750 mesh.

The silica gel can be loaded into the column using a solvent that may be an eluent selected for column chromatography. Docetaxel, dissolved in an organic solvent, such as dichloromethane, acetone, or ethyl acetate, is charged to the column loaded with the silica gel.

Suitable eluents that can be used for the column chromatography include but are not limited to ethyl acetate, isobutyl acetate, n-butyl acetate, n-propyl acetate, isopropyl acetate, n-heptane, n-hexane, cyclohexane, and any combinations thereof.

In an embodiment, a mixture of ethyl acetate and heptane are used as the eluents for column chromatography. The volume percentage of any of the individual solvents in mixture can vary from 1 to 95%.

The eluent fractions containing docetaxel of desired purity are collected and pooled. The solid can be recovered from pure eluent fractions by techniques such as evaporation, atmospheric distillation, distillation under vacuum, agitated thin film drying (“ATFD”), and the like.

In an embodiment, the docetaxel is recovered by distillation of the pure eluent fractions under vacuum. The distillation can be carried out at suitable temperature and vacuum based on the eluents used. In an embodiment, the distillation is carried out until about 80% of the eluent fraction has been removed. In an embodiment, the distillation is carried out to remove at least about 90% of the eluent fraction, or to a minimum volume of the eluent fraction in which docetaxel remains as a slurry and can be stirred and filtered.

In an embodiment, the docetaxel is recovered by distillation of eluent under vacuum to a minimum volume in which docetaxel remains as a slurry, followed by precipitating the solid completely by adding n-heptane, n-hexane, cyclohexane, or combinations thereof.

Docetaxel obtained by the above purification process typically has a purity of not less than about 95%, or not less than about 98%, as determined by HPLC.

3. Purification of step 2 material using acetone and ether.

The docetaxel obtained in the step 2 purification (such as having purity about 95% or higher) can be further purified by recrystallization, which comprises combining a solution of docetaxel in a ketone solvent with an anti-solvent. Docetaxel solutions can be prepared by the dissolution of docetaxel in a ketone solvent. Useful ketone solvents that are used for preparing solutions of docetaxel include, but are not limited to, acetone, methyl isobutyl ketone, methyl ethyl ketone, and the like.

The concentration of docetaxel in the solution is not critical, but the quantity of solvent employed is usually kept to a minimum so as to avoid excessive product losses during the crystallization of solid. The concentration of docetaxel in the solution may generally range from about 0.01 to about 0.5 g/ml.

The solution can be prepared at temperatures ranging from about 25° C. to 100° C., depending on the boiling point of the solvent. Depending on the quantity of solvent taken, docetaxel may dissolve at a temperature of about 25° C. to 35° C., or the solution may need to be heated to elevated temperatures of about 40° C. to 55° C. for dissolution.

Docetaxel can be precipitated from the solution by combining with an anti-solvent. Useful anti-solvents include but are not limited to: alkanes such as n-heptane, n-hexane, cyclohexane and the like; ethers such as diethyl ether, diisopropyl ether, tetrahydrofuran (THF), 1,4-dioxane, dimethoxyethane, methyl tertiary-butyl ether and the like; and combinations thereof.

A decolorizing carbon treatment can be optionally used either at the dissolution temperatures or after cooling the solution to lower temperatures.

Suitable temperatures for solid crystallization can range from about 0° C. to about 80° C., or from about 25° C. to about 35° C.

The obtained precipitate may be separated using conventional techniques known in the art. One skilled in the art may appreciate that there are many ways to separate a solid from the mixture, for example it can be separated by using any techniques such as filtration by gravity or by suction, centrifugation, decantation, and the like. After separation, the solid may optionally be washed with an anti-solvent.

The isolated solid may optionally be further dried. Drying can be suitably carried out using equipment such as a tray dryer, vacuum oven, air oven, fluidized bed drier, spin flash dryer, flash dryer and the like. The drying may be carried out at temperatures of about 35° C. to about 70° C., optionally under reduced pressure. The drying can be carried out for any time periods necessary for obtaining a desired purity, such as from about 1 to about 25 hours, or longer.

The above recyrstallization process can optionally be repeated to get substantially pure docetaxel having a purity greater than or equal to about 99%, or greater than or equal to about 99.5%, as determined using HPLC.

Docetaxel obtained from the purification processes of the present invention contains less than about 0.15% of any individual impurity, the structures of which are given in Table 2 and Table 3.

TABLE 2
Process-Related Docetaxel Impurities

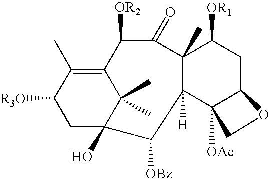

| Impurity | R₁ | R₂ | R₃ |
|---|---|---|---|
| Formula II | —H | —H | —H |
| Formula III | 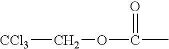 | 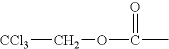 | —H |
| Formula IV | 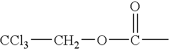 | 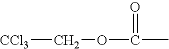 | 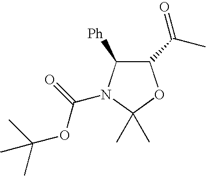 |
| Formula V | —H | —H | 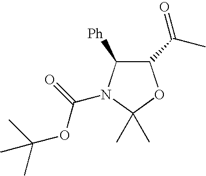 |
| Formula IX | —H | —H | 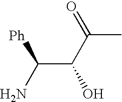 |
| Formula X | 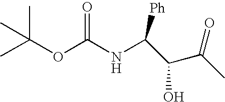 | —H | 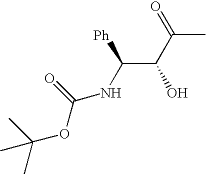 |
| Formula XI | —H | 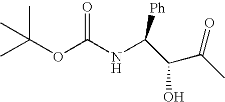 | 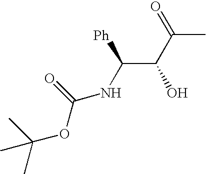 |
| Formula XII | —H | 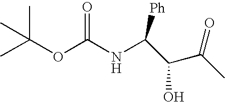 | —H |
| Formula XIII | 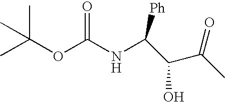 | —H | —H |
| Formula XXII | 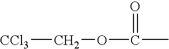 | 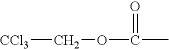 | 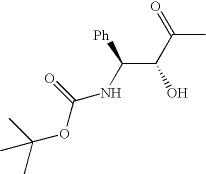 |
| Formula XXIII | 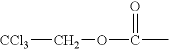 | 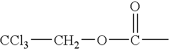 | 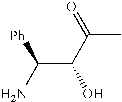 |
| Formula XXV | 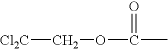 | —H | 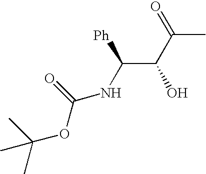 |
| Formula XXVI | —H | 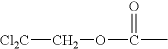 | 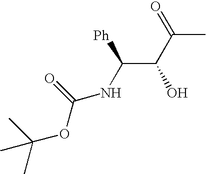 |

TABLE 3
Process-Related Docetaxel Impurities

| Impurity | Structure | Name |
|---|---|---|
| Formula VI | 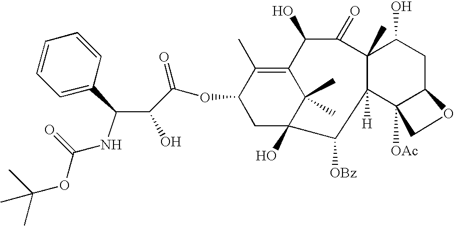 | 7-Epi Docetaxel |
| Formula VII | 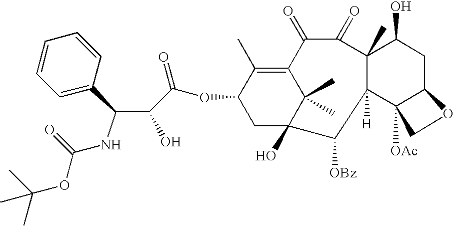 | 10-oxo Docetaxel |
| Formula VIII | 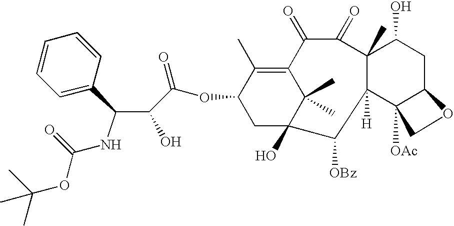 | 10-oxo 7-epi Docetaxel |
| Formula XIV | 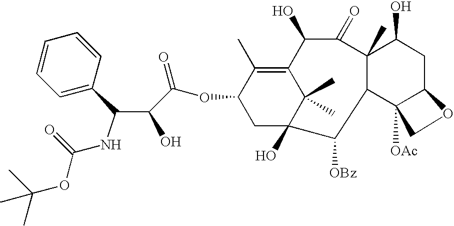 | 2′-Epi Docetaxel |
| Formula XV | 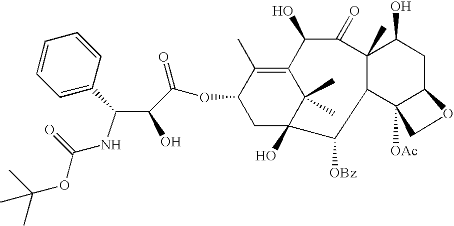 | 2′,3′-Epi Docetaxel |
| Formula XIX | 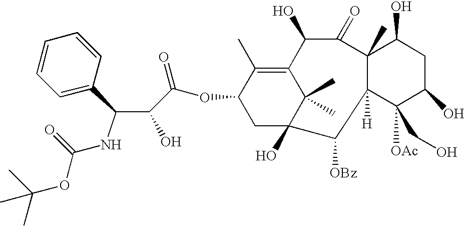 | Oxetane ring opened impurity I |
| Formula XX | 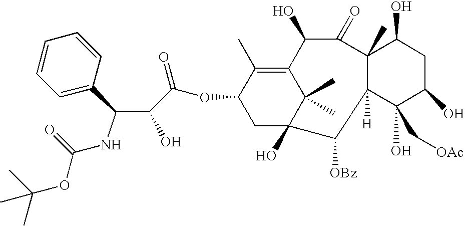 | Oxetane ring opened impurity II |
| Formula XXI | 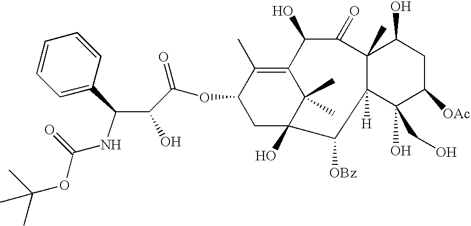 | Oxetane ring opened impurity III |
| Formula XXIV | 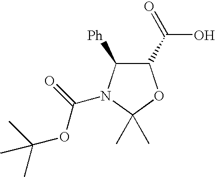 | TSC (Taxane side chain) |

In an embodiment, the present invention provides docetaxel substantially free of its isomers or the related impurities. Docetaxel substantially free of its isomers or the related impurities is obtained by the above process of the present invention.

The term “docetaxel substantially free of its isomers or the related impurities” as used herein shall be understood to mean docetaxel formed with little or no content of the isomers or impurities. The amount of any isomer or impurity of docetaxel resulting from the process of the present invention will be relatively minor, e.g., less than about 0.5 weight percent, or less than about 0.15 weight percent, or less than about 0.05 weight percent, of any isomer or impurity of docetaxel. In particular, docetaxel containing less than about 1%, or less than about 0.5%, of total combinations of the isomers or impurities illustrated in Table 2 and Table 3 may be produced by the process of the present invention.

In another aspect, the present invention provides new crystalline forms of docetaxel.

In one aspect, the present invention relates to crystalline polymorphs of docetaxel, hereinafter referred as Form X and Form XI.

In an embodiment of the present invention, docetaxel crystalline Form X is characterized by the X-ray diffraction pattern with characteristic peaks at 2-theta diffraction angles of about 5.3, 8.9, 10.0, 10.6, 11.2, 12.2, 13.7, 14.1, 15.8, 20.4, 21.2, 21.6, and 21.9, ±0.2 degrees.

In an embodiment, the present invention provides docetaxel crystalline Form X characterized by a DSC thermogram having endotherm peaks about 106° C. and 175° C.

In an embodiment of the present invention, docetaxel crystalline Form X is characterized by a TGA curve corresponding to a weight loss of about 2.6%.

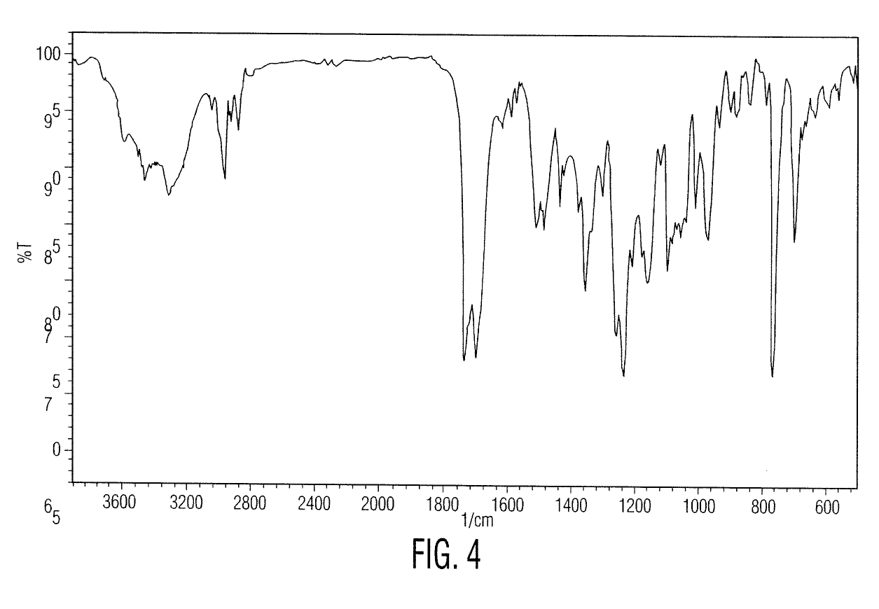

In an embodiment, the present invention provides docetaxel crystalline Form X characterized by an infrared absorption spectrum in a potassium bromide (KBr) pellet substantially as represented by the spectrum of FIG. 4.

In an embodiment of the present invention, there is provided docetaxel Form X, which is substantially pure and free from process related impurities. The crystalline Form X, which is substantially pure contains less than about 0.5%, or less than about 0.1%, of the process related impurities as characterized by HPLC.

In an embodiment, the present invention provides a process for the preparation of crystalline Form X of Docetaxel, comprising the steps of:

a) providing a solution of docetaxel in ethyl acetate;

b) crystallizing a solid from the solution; and

c) drying.

All of the steps for the process for the preparation of crystalline Form X of docetaxel of the present invention are described below.

Step a) involves providing a solution of docetaxel in ethyl acetate.

The solution of docetaxel can be provided by the dissolution of docetaxel in ethyl acetate. Any form of docetaxel is acceptable for providing the solution, such as any amorphous, or hydrated or anhydrous crystalline form of docetaxel in any proportions, obtained by any method; is acceptable for providing the solution.

Docetaxel is mixed with sufficient amount of ethyl acetate to a provide solution of docetaxel at or below the reflux temperature of the ethyl acetate. Optionally, the solution obtained can be filtered to remove any undissolved particles, followed by further processing.

The undissolved particles can be removed using any suitable techniques, including but not limited to filtration, centrifugation, decantation, and other techniques. The solution can be filtered by passing through paper, glass fiber, or other membrane material, or a bed of a clarifying agent such as celite. Depending upon the equipment used and the concentration and temperature of the solution, filtration apparatus may need to be preheated to avoid premature crystallization.

Step b) involves crystallizing the solid from the solution.

Crystallizing the solid from the solution may be performed by methods such as cooling, partial removal of the solvent from the mixture, seeding, adding an anti-solvent to the reaction mixture, or a combination thereof.

In an embodiment, the crystallization is carried out by adding solution of step a) to a suitable anti-solvent to the solution. Suitable anti-solvents that can be used include but are not limited to hydrocarbons such as n-hexane, n-heptane, cyclohexane and the like. The volume of anti-solvent can vary from about 3 to about 10 times the volume of the docetaxel solution. The docetaxel solution can be combined with the anti-solvent for a sufficient period of time, which may range from about 15 minutes to 2 hours, or longer, to effect the desired crystallization. Suitable temperatures for crystallization may range from about 20° C. to about 50° C. The obtained suspension may then be stirred for about 30 minutes to 5 hours, or longer, depending upon the desired precipitation.

The obtained precipitate may be isolated using conventional techniques known in the art. One skilled in the art may appreciate that there are many ways to separate a solid from the mixture, for example it can be separated by using any techniques such as filtration by gravity or by suction, centrifugation, decantation, and the like. After separation, the solid may optionally be washed with a suitable solvent such as n-hexane, n-heptane, or cyclohexane.

Step c) involves drying.

The isolated solid may optionally be further dried. Drying can be suitably carried out in a tray dryer, vacuum oven, air oven, fluidized bed drier, spin flash dryer, flash dryer and the like. The drying may be carried out at temperatures of about 35° C. to about 70° C., optionally under reduced pressure. The drying can be carried out for any time periods necessary for obtaining a desired purity, such as from about 1 to about 25 hours, or longer.

In another embodiment the invention provides docetaxel crystalline Form XI characterized by an XRPD pattern with characteristic peaks at diffraction angles 2-theta of about 4.4, 4.5, 7.0, 8.0, 8.7, 9.1, 11.0, 11.4, 12.3, 12.5, 13.5, 14.1, 15.4, 16.5, 16.9, 17.4, 18.4, 19.5, and 20.4, ±0.2 degrees.

In another embodiment, the present invention provides docetaxel crystalline Form XI characterized by a DSC thermogram having endotherm peaks at about 66° C. and 161° C.

In yet another embodiment of the present invention, docetaxel crystalline Form XI is characterized by a TGA curve corresponding to a weight loss of about 2%.

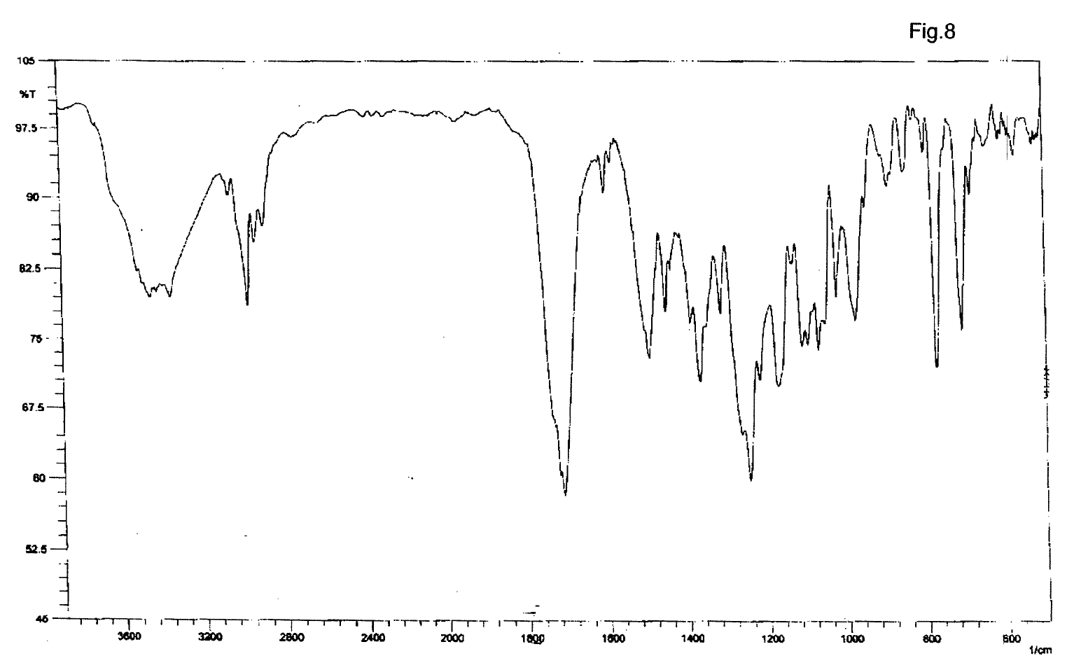

In an embodiment, the present invention provides docetaxel crystalline Form XI characterized by an IR absorption spectrum in a potassium bromide (KBr) pellet substantially as represented by the spectrum of FIG. 8.

In a further embodiment of the present invention, there is provided docetaxel Form XI, which is substantially pure. The crystalline Form XI of the present invention may contain less than about 0.5%, or less than about 0.1%, of process-related impurities, as characterized by HPLC.

In an embodiment, the present invention provides processes for the preparation of crystalline Form XI of Docetaxel, an embodiment comprising:

a) providing a solution of docetaxel in acetone;

b) precipitating a solid by adding an ether; and

c) drying.

All of the steps the preparation of crystalline Form XI of docetaxel of the present invention are separately described below.

Step a) involves providing a solution of docetaxel in acetone.

The solution of docetaxel can be provided by dissolving docetaxel in acetone. Any form of docetaxel is acceptable for providing the solution, such as any amorphous, or hydrated or anhydrous crystalline form of docetaxel. Solutions of docetaxel can be provided in acetone at temperatures from about 25° C. to about 55° C., or about 40° C. to about 45° C.

Any undissolved particles can be removed suitably by filtration, centrifugation, decantation, and other techniques.

Step b) involves precipitating the solid by adding an ether.

The docetaxel solution is combined with ether for precipitation. Addition of docetaxel solution to ether can be carried out over a period of about 30 minutes to 1 hour, or longer. The volume of ether can vary from about 2 to 8 times the volume of the docetaxel solution. The obtained solution can be stirred for about 30 minutes to about 2 hours, or longer, to increase the precipitation.

Suitable ethers that can used in the process of the present invention include, but are not limited to, diethyl ether, diisopropyl ether, tetrahydrofuran (THF), 1,4-dioxane, dimethoxyethane, and methyl tertiary-butyl ether.

The obtained solid precipitate may be isolated using conventional techniques known in the art. One skilled in the art may appreciate that there are many ways to separate a solid from the mixture, for example it can be separated using any techniques such as filtration by gravity or by suction, centrifugation, decantation, and the like. After separation, the solid may optionally be washed with a suitable solvent such as n-hexane, n-heptane, or cyclohexane.

Step c) involves drying the solid.

The wet solid may optionally be further dried. Drying can be suitably carried out in a tray dryer, vacuum oven, air oven, fluidized bed drier, spin flash dryer, flash dryer and the like. The drying may be carried out at temperatures of about 35° C. to about 70° C., optionally under reduced pressure. The drying can be carried out for any time periods necessary for obtaining a desired purity, such as from about 1 to about 40 hours, or longer.

In yet another aspect of the present invention there are provided processes for preparing docetaxel trihydrate, an embodiment comprising:

a) providing a mixture of docetaxel in water;

b) maintaining the mixture at 25-30° C., under stirring; and

c) drying.

All the steps for process for the preparation of docetaxel trihydrate of the present invention are separately described below.

Step a) involves providing a mixture of docetaxel in water

Docetaxel is mixed with an amount of water to provide a suspension. Docetaxel is mixed with sufficient amount of water to provide a suspension of docetaxel at or below the reflux temperature of the water.

The starting material, which can be used for the preparation of a trihydrate polymorphic form of the present invention, can be either crude docetaxel or pure docetaxel obtained by the purification process disclosed in the present invention or by any method known in the art. The starting material can be of any polymorphic form, such as amorphous, or hydrated or anhydrous crystalline form of docetaxel or mixtures of amorphous and crystalline forms of docetaxel in any proportions, obtained by any method.

Step b) involves maintaining the mixture at 25-30° C., under stirring.

The mixture of step a) is maintained under stirring for a sufficient time to affect adequate product formation. In embodiments, the mixture is maintained from about 30 minutes to about 5 hours, or longer, depending upon the desired extent of crystallization. Suitably, the precipitated slurry can be maintained at temperatures from about 20° C. to about 100° C., or about 25° C. to about 35° C.

The obtained precipitate may be isolated using conventional techniques known in the art. In embodiments, the compound is isolated by filtration using suction. After separation, the solid may optionally be washed with water.

Step c) involves drying.

The wet solid obtained in the above step may optionally be dried. Drying can be suitably carried out in a tray dryer, vacuum oven, air oven, fluidized bed drier, spin flash dryer, flash dryer and the like. The drying may be carried out at temperatures of about 25° C. to about 70° C. for any time periods necessary for obtaining desired purity, optionally under reduced pressure. In embodiments, drying is carried out at 25-40° C. under a vacuum of 680-720 mm of Hg, until docetaxel trihydrate with the desired moisture level is obtained.

Docetaxel trihydrate obtained the process of the present invention is characterized by its XRPD pattern with characteristic peaks at diffraction angles 2-theta of about 4.5, 7.3, 8.9, 10.5, 11.2, 12.4, 12.7, 13.1, 13.6, 14.1, 15.4, 16.6, 17.2, 17.8, 18.5, 19.4, and 19.9, ±0.2 degrees.

Further, docetaxel trihydrate obtained the process of the present invention can be characterized by:

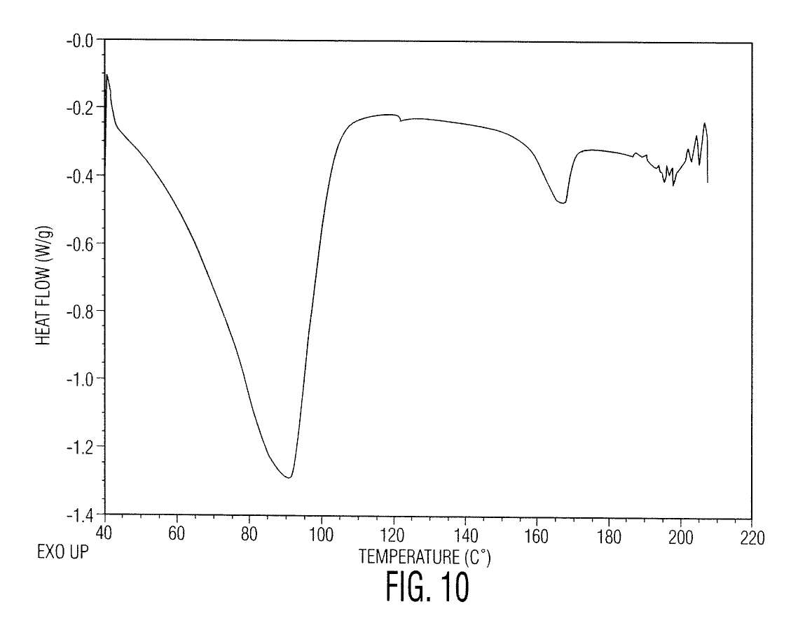

a. its DSC thermogram as represented by FIG. 10;

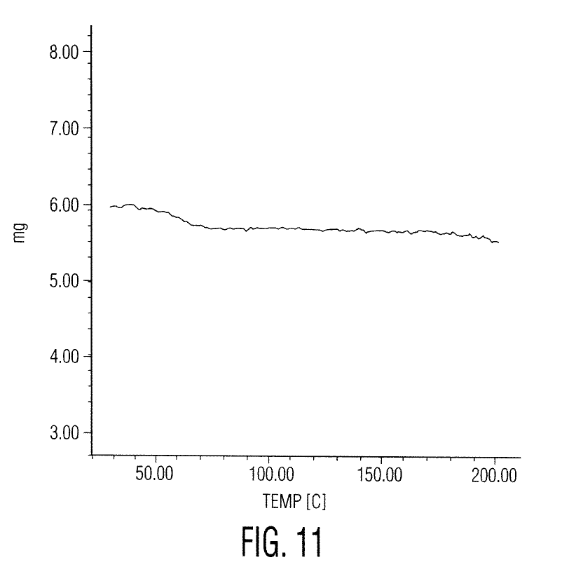

b. its TGA curve as represented by FIG. 11; and

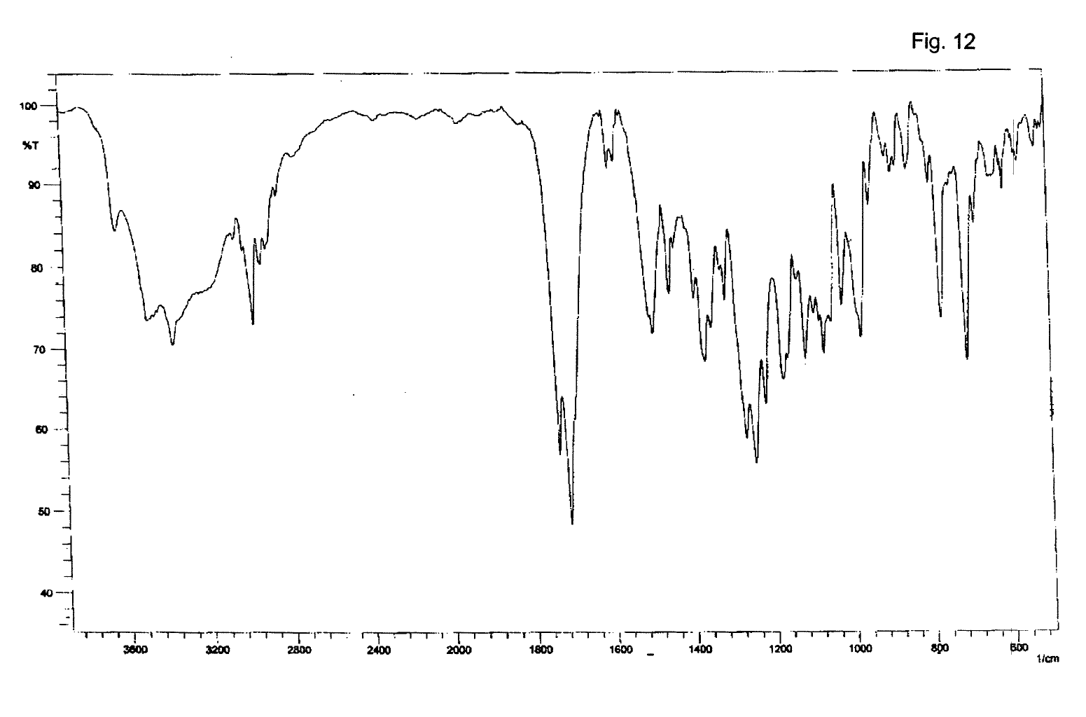

c. its IR absorption spectrum in a potassium bromide (KBr) pellet as represented by the spectrum of FIG. 12.

Yet another aspect of the present invention provides pharmaceutical compositions of docetaxel of the present invention, which comprises at least two components system, where:

a) component 1 comprises a therapeutically effective amount of a docetaxel, polyethylene glycol 660 12-hydroxystearate, and an alcohol; and

b) component 2 comprises a solution of polyethylene glycol 660 12-hydroxystearate and an alcohol.

In a further embodiment, parenteral docetaxel compositions of the present invention are substantially free from polysorbates, and may optionally contain a polyoxyethylated castor oil.

In another embodiment, the docetaxel compositions of the present invention, upon dilution with aqueous fluid, are administered parenterally to a mammal in need of docetaxel therapy, wherein such therapy exhibits reduced toxic manifestations associated with polysorbates.

Another aspect of the present invention provides a kit suitable for reconstitution using an aqueous dilution fluid for parenteral administration, wherein the kit comprises a two-component pharmaceutical composition, wherein

a) component 1 comprises a therapeutically effective amount of docetaxel, polyethylene glycol 660 12-hydroxystearate, and an alcohol; and

b) component 2 comprises a solution of polyethylene glycol 660 12-hydroxystearate, and an alcohol.

In an embodiment, the two components of the present invention can be mixed in specific proportions to obtain a “pre-mix,” which is further diluted with an aqueous fluid for parenteral administration.

Docetaxel used for the said pharmaceutical compositions can be of any form such as amorphous, anhydrous or hydrated crystalline forms of docetaxel, or mixtures of amorphous and crystalline forms of docetaxel in any proportions, obtained by any method. In embodiments, a docetaxel hydrate is used for the pharmaceutical compositions. In other embodiments, docetaxel trihydrate is used for the pharmaceutical compositions.

“Components” in the context of the present invention refers to compositions in separate containers including ampoules, syringes, dual chamber syringes, vials and the like. The two containers for the compositions of the present invention may be similar or different, and may be made of materials such as metals like steel, non-metals like various grades of glass and plastics such as polyethylene, and the like, and combinations thereof. In some embodiments, the components can be in separate containers or discrete spaces within a single container. An example of a useful container is a two-compartment cartridge for use in a syringe, where components of the compartments are mixed in the cartridge as the syringe plunger first is advanced, and then can be ejected from the cartridge as the plunger is further advanced.

For ease of understanding, a representative two-component composition of the present invention may comprise a “stock solution” that contains docetaxel (component 1), and a “diluent” that does not contain docetaxel (component 2).

In an embodiment, the two components (viz., stock solution and diluent) of the present invention can be mixed in specific proportions to obtain a “pre-mix”, which is further diluted with aqueous fluid for parenteral administration. The volume ratios of stock solution to diluent in the premix typically vary between about 1:1 and about 1:5, so as to obtain a desired administrable strength of docetaxel upon dilution with aqueous fluid.

“Therapeutically effective amount” (used interchangeably with “pharmaceutically effective amount”) refers to the amount of a drug (e.g., docetaxel) that is effective to treat diseases or disorders (e.g., cancer), at a reasonable benefit/risk ratio applicable to any medical treatment.

The term “therapy” or “treatment” as used herein refers to management and care of a patient for the purpose of combating a condition, such as a disease or a disorder. The term is intended to include the full spectrum of treatments for a given condition from which the patient is suffering, such as administration of docetaxel to alleviate the symptoms or complications, to delay the progression of the disease, disorder or condition, to alleviate or relief the symptoms and complications, and/or to cure or eliminate the disease, disorder or condition as well as to prevent the condition. The patient to be treated is preferably a mammal, in particular a human, and may also include animals such as rats, mice, dogs, cats, cows, sheep and swine.

Polyethylene glycol 660 12-hydroxystearate is commercially available as SOLUTOL® HS-15 (marketed by BASF, Germany). SOLUTOL® HS-15 is a non-ionic solubilizer that becomes liquid at about 30° C., and is suitable for injectable solutions. It has a polyglycol ester of 12-hydroxystearic acid as a hydrophobic component (70%) and polyethylene glycol as a hydrophilic component (30%). Solutol® HS15 dissolves in water, ethanol and 2-propanol to form clear solutions. Aqueous solutions of Solutol® HS15 can be sterilized by heating them to 120° C. SOLUTOL® HS-15 is less toxic, as compared to polyoxyethylated castor oils (commercially available as Cremophor®) and polysorbates.

Surprisingly, it has been observed that the two-component docetaxel compositions of the present invention yield a stable product, which can be reconstituted with an aqueous fluid before parenteral administration to a mammal in need thereof. Such reconstituted formulations exhibit desirable physical and chemical stability.

Typically, the pharmaceutical compositions of the present invention comprise the following concentrations of ingredients: docetaxel about 1% to about 6%, or about 2% to about 5%, by weight; polyethylene glycol 660 12-hydroxystearate about 30% to about 60%, or about 40% to about 50%, by weight; and alcohol about 30% to about 80%, or about 35% to about 75%, by weight.

In some embodiments, polyethylene glycol 660 12-hydroxystearate can be used in combination with other surfactants such as polyoxyethylated castor oils. Although polyoxyethylated castor oils are known to cause hypersensitivity reactions upon parenteral administration, partly replacing it with polyethylene glycol 660 12-hydroxystearate may reduce such hypersensitivity reactions. The weight ratios of polyethylene glycol 660 12-hydroxystearate to polyoxyethylated castor oil may range from about 1:0.1 to about 1:5, or about 1:0.5 to about 1:2, in the pre-mix.

In an embodiment, the pharmaceutical compositions of the present invention comprise weight ratios of docetaxel to polyethylene glycol 660 12-hydroxystearate in the range of about 1:10 to about 1:40, or about 1:15 to about 1:30.

Particularly, a first component of the present invention comprises docetaxel in the range of about 20 to about 40 mg/mL, polyethylene glycol 660 12-hydroxystearate in the range of about 600 to about 650 mg/mL, and alcohol in the range of about 300 to about 350 mg/mL. The mixture of this component are frequently adjusted to pH values about 3 to about 4 using citric acid. A second component of the present invention comprises polyethylene glycol 660 12-hydroxystearate in the range of about 200 to about 250 mg/mL and alcohol in the range of about 300 to about 350 mg/mL.

In another embodiment, the second component of the present invention typically comprises water in the concentration range of about 20% w/w to about 60% w/w, or about 30% w/w to about 50% w/w, of the total component.

In the context of the present invention, “alcohol” refers to compounds such as ethanol, propylene glycol, glycerol, glycofurol, polyethylene glycol, etc., and mixtures thereof.

The compositions of the present invention may optionally contain pharmaceutically acceptable additives such as pH modifiers, buffering, chelating, complexing and solubilizing agents, antioxidants and antimicrobial preservatives, suspending and/or viscosity modifying agents, tonicity modifying agents, and other biocompatible materials or therapeutic agents.

An aspect of the present invention provides use of a co-solvent or solubilizing agent in the compositions to solubilize other components of the system. Non-limiting examples of co-solvents, in the context of the present invention, include substances such as ethanol, propylene glycol, glycerol, glycofurol, polyethylene glycol, diethylene glycol monoethyl ether (TRANSCUTOL®), and mixtures thereof.

“Antioxidant” as used herein includes metal ion chelators and/or reducing agents. A metal ion chelator functions as an antioxidant by binding to metal ions and thereby reduces the catalytic effect of metal ions on oxidation reactions of the drug, oil, or phospholipid components. Metal chelators useful in this invention include, but are not limited to, EDTA, glycine and citric acid or salts thereof. Non-limiting examples of antioxidants also include natural vitamin E, vitamin-E succinate, ascorbic acid, sodium metabisulfite, amino acids, flavones, monothioglycerol, L-cysteine, thioglycolic acid and mixtures thereof. Such antioxidants may be used in concentration ranges of about 0.1 to 15% w/v, or about 0.5 to 5% w/v.

Non-limiting examples of pH modifiers and stabilizers include citric acid, tartaric acid, succinic acid, glutamic acid, ascorbic acid, lactic acid, acetic acid, malic acid, maleic acid, and sodium salts thereof, sodium hydroxide, sodium carbonate, sodium bicarbonate, tris buffer, meglumine, amino acids and mixtures thereof. Such pH modifiers and stabilizers maintain a desired pH between about 1 and 8, or between about 2.5 and 5.5, in the composition.

In certain embodiments, the present compositions are both chemically and physically stable. A pharmaceutical composition is “chemically stable” if the drug in the composition is not substantially chemically degraded after storage under commercially appropriate conditions. A pharmaceutical composition is “physically stable” if it is stored under commercially appropriate conditions without evidence of precipitation, separation, or aggregation.

In some embodiments, the dispersions obtained after suitable dilution of taxane compositions of the present inventions may be parenterally administered to a subject. “Parenteral” includes any mode of administration that does not go through the digestive tract, but excludes trans-membrane delivery such as skin patches. Parenteral administration most commonly refers to injections or infusions into blood vessels. In certain embodiments, the mode of administration of the present dispersions is by intravenous, intra-arterial, intrathecal, intraperitoneal, intratumoral, intra-articular, intramuscular, subcutaneous, and the like.

In an embodiment, a process for preparation of a pharmaceutical composition of the present invention comprises:

1. For component 1, mixing alcohol and polyethylene glycol 660 12-hydroxystearate, adjusting the pH to about 3 to about 4 with citric acid, and dissolving docetaxel in this mixture to get a solution; and

2. For component 2, mixing alcohol, water and polyethylene glycol 660 12-hydroxystearate.

The compositions of individual components are frequently aseptically filtered, such as through a 0.22 μm filter membrane, and filled into containers of desired capacities.

The pharmaceutical compositions of the present invention may be stored at about 2° C. to 8° C., or up to and including temperatures that generally do not exceed normal room temperatures.

In an embodiment, the pharmaceutical compositions of the present invention are diluted with an aqueous fluid, including water, various buffer solutions having different pH values, parenteral infusion fluids, and other such media. Typically used parenteral infusion fluids include 5% dextrose solution, 0.9% sodium chloride solution (normal saline), Ringer's lactate, mannitol infusion fluid, sucrose infusion fluid, plasma volume expanders, and mixtures thereof, and will have docetaxel concentrations in the infusion fluid ranging between about 0.1 mg/mL and about 1 mg/mL, or between about 0.3 mg/mL and about 0.8 mg/mL, at the time of parenteral administration. This diluted docetaxel composition may be administered parenterally to a mammal in need thereof using suitable infusion bags and administration sets, as are well known to persons skilled in the art.

The pharmaceutical compositions according to the instant invention may be used for the treatment of various disease states like cancers, tumors, Kaposi's sarcoma, malignancies, uncontrolled tissue or cellular proliferation secondary to tissue injury, and any other disease conditions responsive to taxanes such as paclitaxel and docetaxel, or prodrugs, analogs and derivatives of the foregoing. Among the types of carcinoma, which may be treated particularly effectively with docetaxel, other taxanes, and their prodrugs and derivatives, are hepatocellular carcinoma and liver metastases, cancers of the gastrointestinal tract, pancreas, prostate and lung, and Kaposi's sarcoma. Generally, the compositions of the present invention, either alone or in combination with other drugs, are useful for treatment of tumors in breast, lung, stomach, head, neck and prostate tissues, esophageal neoplasms, and any other such tumors in mammals.

In an embodiment present invention relates to pharmaceutical compositions wherein docetaxel may be replaced with any of the other taxanes such as but not limited to paclitaxel, or its pharmaceutically acceptable analogs, polymorphs, solvates or mixtures thereof, process for preparing the same and their method of use.

The following examples are provided only to further illustrate certain specific aspects and embodiments of the invention, and should not be construed as limiting the scope of the invention in any manner.

### Example 1

### Preparation of Docetaxel

Step A): Preparation of the Compound of Formula A.

Acetic acid (20 L) and the compound of Formula B (2 Kg) were charged into a clean and dry round bottom flask with stirring. The solution was stirred for about 10 minutes at about 25-30° C. and then methanol (20 L) was charged to the solution. Zinc dust (0.871 Kg) was charged to the solution and then heated to about 55-60° C. The mixture was stirred for about 30 minutes at about 55-60° C. The reaction mixture was filtered through a Hyflow (flux-calcined diatomaceous earth) bed and the bed was washed with methanol (2 L). The filtrate was slowly added to water (140 L) and stirred for about 1 hour at about 25-30° C. The suspension was filtered under vacuum and the solid was washed with water (4 L). The solid was dissolved in ethyl acetate (20 L) and then the ethyl acetate layer was washed with water (10 L). The ethyl acetate layer was concentrated until the volume was between 8 L and 12 L, at about 50° C. under vacuum. The mass was slowly transferred to a vessel containing n-heptane (about 5 times the volume of the concentrated mass) at about 25-30° C., and then stirred for about 1 hour. The solid obtained was filtered, washed with n-heptane (2 L) and dried at about 45-50° C. for about 4 hours to afford the title compound. Yield: 0.87 Kg. Purity: 93.39% by HPLC.

Step B): Preparation of Docetaxel of Formula I.

Formic acid (8.7 L) was placed into a round bottom flask and cooled to 15-20° C. The compound of Formula A (0.87 Kg) was added and stirred for 90 minutes at 15-20° C. The solution was concentrated at 45-50° C. under a vacuum of 680 to 720 mm Hg over about one hour, to about 1.7 L. Water (8.7 L) and ethyl acetate (8.7 L) were charged to the mass and then stirred for 10-15 minutes. The organic layer was separated and ethyl acetate (8.7 L) was charged to the mass and then stirred for 10-15 minutes. The organic layer was separated. Ethyl acetate (8.7 L) was charged to the mass and pH was adjusted to 7.5-8.5 with solid NaHCO₃ (˜1.16 Kg) at 25-30° C. over 10 minutes. Di-t-butyl dicarbonate (0.35 Kg) was charged to the mass at 25-30° C. and stirred for 60 minutes. Separated the organic layer from the mass and concentrated the reaction mass at 45-50° C. under high vacuum to 2.6 L to 4.3 L. Cooled the mass to 25-30° C. and slowly charged the mass to a vessel containing n-heptane (21.75 L). Stirred the mass for 60 minutes at 25-30° C., filtered and washed the wet cake with n-heptane and dried at 45-50° C. for 4 hours under high vacuum. Yield: 0.71 Kg. Purity: 80.4% by HPLC.

Step C) Purification.

Purification 1: Purification of docetaxel from acetonitrile-diisopropyl ether.

Acetonitrile (1.4 L) and docetaxel (0.71 Kg) obtained from step B) were charged into a clean and dry round bottom flask. The mixture was stirred for about 10 minutes at 25-30° C. Diisopropyl ether (7.0 L) was charged to the mixture and stirred for 90 minutes at 25-30° C. The solid obtained was filtered, washed with diisopropyl ether (1.4 L) and suction dried for about 30 minutes under a vacuum of 680 mm Hg to afford the title compound. Yield: 0.43 Kg. Purity: 92.3% by HPLC.

Purification 2: Purification of docetaxel using column chromatography.

A column was packed with silica gel (12.5 Kg) in 20% of ethyl acetate in n-heptane (34.3 L). Docetaxel (0.43 Kg, 92.3% purity) was dissolved in dichloromethane (1.0 L) and charged to the column. The column was eluted with a mixture of ethyl acetate and n-heptane (25.8 L of 20% ethyl acetate in n-heptane, 25.8 L of 40% ethyl acetate in n-heptane, and 129 L of 45% ethyl acetate in n-heptane). After elution of about 10-15 L, a purified fraction of 165 L was collected. The purified fraction was concentrated at about 45-50° C. under a vacuum of 680 mm Hg to a volume in the range of 4 L to 12 L and cooled to about 25-30° C. Stirred the reaction mass for 60 minutes at 25-30° C., filtered under vacuum, washed the wet cake with n-heptane (1.0 L) and the solid material was dried for about 4 hours at 45-50° C. under high vacuum to give purified docetaxel. Yield: 0.3 Kg (70-80%). Purity: 99.09% by HPLC.

Impurities: Formula XX 0.1%, Formula IX 0.1%, Formula XIV not detected, Formula VII 0.05%, Formula VI not detected, and Formula VIII not detected.

Step D): Final Purification of Docetaxel.

Acetone (6 L) and docetaxel (0.3 Kg) as obtained from step C) were charged into a clean and dry round bottom flask. The mixture was heated to about 45-50° C. to get a solution, cooled to 25-30° C., and filtered through a 0.4 μm filter paper. Diisopropyl ether (20 L) was charged into another round bottom flask and the filtrate obtained was added over a period of about 30-45 minutes at 25-30° C. The suspension was stirred for about 90 minutes at 25-30° C. and then filtered. The wet solid obtained was washed with diisopropyl ether (0.6 L) and dried at a temperature of about 55-60° C. for about 24 hours under a vacuum of 680 mm Hg to afford the title compound. Yield: 0.234 Kg. Purity: 99.74% by HPLC.

Impurities: Formula XX 0.07%, Formula IX 0.09%, Formula XIV not detected, Formula VII 0.04%, Formula V10.06%, and Formula VIII not detected.

### Example 2

### Purification of Docetaxel Using Column Chromatography

A column was packed with silica gel (625 g) in 20% of ethyl acetate in n-heptane (2 L). Docetaxel (25 g) was dissolved in ethyl acetate (50 ml) and charged to the column. The column was eluted with a mixture of ethyl acetate and n-heptane (2 L of 20% ethyl acetate in heptane and 20 L of 50% ethyl acetate in heptane). After elution of 11 L, a purified fraction of 8.5 L was collected. The purified fraction was concentrated to about 50 ml at a temperature of about 47° C. under a vacuum of 680 mm Hg and cooled to about 25° C. to 30° C. The concentrated mass was added to n-heptane (250 ml) and stirred for about 15 to 30 minutes. The residue was filtered and washed with n-heptane (25 ml), and the solid material was dried for about 2 hours at about 50° C. to afford 16.2 g of title compound. Purity: 94.47% by HPLC.

### Example 3

### Purification of Docetaxel

Acetone (1370 ml) and docetaxel (137 g) were charged into a clean and dry round bottom flask. The mixture was heated to about 45° C. and then stirred for about 30 minutes. The solution was filtered and the filtrate was cooled to about 27° C. Diisopropyl ether (4110 ml) was charged into another reactor and the filtrate was added over a period of about 30 minutes. The suspension was stirred for about 1.5 hours and then filtered. The solid was washed with diisopropyl ether (275 ml) and dried at a temperature of about 60° C. for about 4 days under a vacuum of 680 mm Hg to afford 92 g of the title compound. Purity: 99.38% by HPLC.

### Example 4

### Preparation of Crystalline Form X of Docetaxel

Anhydrous docetaxel (2 g) was dissolved in ethyl acetate (20 ml) at a temperature of about 25° C. to 30° C. The solution was then filtered through a 0.4 μm filter paper. n-Heptane (100 ml) was charged into a round bottom flask and the filtrate was added to the n-heptane over a period of about 30 to 45 minutes. The suspension was stirred for about 60 minutes at a temperature of about 25° C. to 30° C. The solid was collected by filtration, washed with n-heptane (20 ml) and suction dried. The solid was dried under a vacuum for about 36 hours at a temperature of about 50° C. to afford 1.75 g of title compound.

XRPD pattern peaks: 5.3, 8.9, 10.0, 10.6, 11.2, 12.2, 13.7, 14.1, 15.8, 20.4, 21.2, 21.6, and 21.9.±0.2 degrees 2Θ.

### Example 5

### Preparation of Docetaxel Crystalline Form X

Docetaxel trihydrate (2 g) was dissolved in ethyl acetate (10 ml) at a temperature of about 25° C. to 30° C. and the solution was filtered through a 0.4 μm filter paper. n-Heptane (50 ml) was charged into a round bottom flask and the filtrate was added to n-heptane over a period of about 30 to 45 minutes. The suspension was stirred for about 60 minutes at a temperature of about 25° C. to 30° C. The solid was collected by filtration, washed with n-heptane (10 ml) and suction dried. The solid was dried under a vacuum for about 36 hours at a temperature of about 50° C. to afford 1.8 g of title compound.

XRPD pattern peaks: 5.3, 8.9, 10.0, 10.6, 11.2, 12.2, 13.7, 14.1, 15.8, 20.4, 21.2, 21.6, and 21.9, ±0.2 degrees 2Θ.

### Example 6

### Preparation of Docetaxel Crystalline Form X

Amorphous docetaxel (2 g) was dissolved in ethyl acetate (10 ml) at a temperature of about 25° C. to 30° C. The solution was filtered through a 0.4 μm filter paper. n-Heptane (50 ml) was charged into a round bottom flask and the filtrate was added to n-heptane over a period of about 30 to 45 minutes. The suspension was stirred for about 60 minutes at a temperature of about 25° C. to 30° C. The solid was collected by filtration, washed with 10 ml of n-heptane and suction dried. The solid was dried under a vacuum for about 36 hours at a temperature of about 50° C. to afford 1.76 g of title compound.

XRPD pattern peaks: 5.3, 8.9, 10.0, 10.6, 11.2, 12.2, 13.7, 14.1, 15.8, 20.4, 21.2, 21.6, and 21.9, ±0.2 degrees 2Θ.

### Example 7

### Preparation of Docetaxel Crystalline Form XI

Anhydrous docetaxel (2 g) was dissolved in acetone (20 ml) at a temperature of about 45° C. and then the solution was cooled to 30° C. Diisopropyl ether (60 ml) was charged into a clean and dry round bottom flask. The docetaxel solution was added to the diisopropyl ether over a period of about 60 minutes. The suspension was stirred for about 90 minutes and then filtered. The solid was washed with diisopropyl ether (10 ml) and then suction dried under a vacuum of 600 mm Hg for about 30 minutes. The obtained solid was dried over a period of about 36 hours to afford 1.68 g of title compound.

XRPD pattern peaks: 4.4, 4.5, 7.0, 8.0, 8.7, 9.1, 11.0, 11.4, 12.3, 12.5, 13.5, 14.1, 15.4, 16.5, 16.9, 17.4, 18.4, 19.5, and 20.4, ±0.2 degrees 2Θ.

### Example 8

### Preparation of Docetaxel Crystalline Form XI

Amorphous docetaxel (2 g) was dissolved in acetone (20 ml) at a temperature of about 45° C. and the solution was cooled to about 30° C. Diisopropyl ether (60 ml) was charged into a clean and dry round bottom flask. The docetaxel solution was added to the diisopropyl ether over a period of about 60 minutes. The suspension was stirred for about 90 minutes and then filtered. The solid was washed with diisopropyl ether (10 ml) and then suction dried under a vacuum of 600 mm Hg for a period of about 30 minutes. The obtained solid was dried over a period of about 36 hours to afford 1.77 g of title compound.

XRPD pattern peaks: 4.4, 4.5, 7.0, 8.0, 8.7, 9.1, 11.0, 11.4, 12.3, 12.5, 13.5, 14.1, 15.4, 16.5, 16.9, 17.4, 18.4, 19.5, and 20.4, ±0.2 degrees 2Θ.

### Example 9

### Preparation of Crystalline Docetaxel Trihydrate

Demineralized water (40 ml) and anhydrous docetaxel (2 g) were charged into a round bottom flask at a temperature of about 25° C. to 30° C. The suspension was stirred for about 60 minutes and filtered. The filtered solid was washed with demineralized water (10 ml) and then suction dried under a vacuum of 650-700 mm Hg for about 30-60 minutes. The wet solid was dried under a vacuum for about 2-3 hours at a temperature of about 25° C.-30° C. to afford 2 g of title compound.

XRPD pattern peaks: 4.5, 7.3, 8.9, 10.5, 11.2, 12.4, 12.7, 13.1, 13.6, 14.1, 15.4, 16.6, 17.2, 17.82, 18.5, 19.4, and 19.9, ±0.2 degrees 2Θ.

### Example 10

### Preparation of Crystalline Docetaxel Trihydrate

Demineralized water (40 ml) and amorphous docetaxel (2 g) were charged into a round bottom flask at a temperature of about 25° C. to 30° C. The suspension was stirred for about 60 minutes and filtered. The filtered solid was washed with demineralized water (10 ml) and then suction dried under a vacuum of 650-700 mm Hg for about 30-60 minutes. The wet solid was dried under a vacuum for about 2-3 hours at a temperature of about 25° C.-30° C. to afford 2 g of title compound.

XRPD pattern peaks: 4.5, 7.3, 8.9, 10.5, 11.2, 12.4, 12.7, 13.1, 13.6, 14.1, 15.4, 16.6, 17.2, 17.82, 18.5, 19.4, and 19.9, ±0.2 degrees 2Θ.

### Example 11

### Two-Component Docetaxel Compositions and Pre-Mix

| | mg/mL | | |
|---|---|---|---|
| Ingredient | Component 1 | Component 2 | Pre-mix* |
| Docetaxel trihydrate | 40 | — | 10 |
| SOLUTOL ® HS-15 | 618 | 206 | 309 |
| Ethanol | 319.6 | 319.6 | 319.6 |
| Water | — | 400 | 300 |
| Citric acid anhydrous | q.s. to pH | — | — |
| | 3.55 | | |

*A 2:6 v/v ratio of component 1 to component 2.

Manufacturing process:

For Component 1:

1. Ethanol and Solutol HS 15 were mixed well.

2. The pH of the above mixture was adjusted using citric acid anhydrous.

3. Docetaxel was dissolved in the mixture of step 2.

4. Solution of step 3 was filtered through a 0.22 μm filter membrane and filled into vials.

For Component 2:

1. Ethanol, water for injection and Solutol HS 15 were mixed well.

2. Solution of step 1 was filtered through a 0.22 μm filter membrane and filled into vials.

Dilution for two-component composition (“pre-mix”) of docetaxel:

1. The entire contents of a component 2 vial were withdrawn and injected into a component 1 vial, and mixed well by gentle shaking.

2. The mixture of step 1 was diluted with 250 mL of 0.9% sodium chloride solution and mixed by gentle shaking.

Physical properties of one-component compositions of docetaxel and their diluted formulations.

| Parameter | Example 1 |
|---|---|
| Syringeability of component 2* | Grade 2 |
| Syringeability of pre-mix* | Grade 2 |
| pH of pre-mix | 4.25 |
| pH of diluted formulation | |
| Initial | ND** |
| After 3 hours | ND** |
| After 24 hours | 3.92 |
| Visually observed precipitation of | No precipitation (clear solution) |
| diluted formulation | observed over 24 hours |

*Syringeability was evaluated based on grading system as described in Comparative Example 1.
** Not done.

### Comparative Examples 12(A-C)

### One-Component Docetaxel Compositions

| | mg/mL | | |
|---|---|---|---|
| | Comparative | Comparative | Comparative |
| Ingredient | Example A | Example B | Example C |
| Docetaxel trihydrate | 40 | 20 | 20 |
| Polyethylene glycol 660 | 618 | 618 | 600 |
| 12-hydroxystearate | | | |
| (SOLUTOL ® HS-15)* | | | |
| Ethanol | 319.6 | 319.6 | 331 |
| Citric acid anhydrous | q.s. to | q.s. to | q.s. to |
| | pH 3.5 | pH 3.7 | pH 3.9 |

*Marketed by BASF, Germany.

Manufacturing process:

1. Ethanol and Solutol HS 15 were mixed well.

2. The pH of the mixture was adjusted using citric acid anhydrous.

3. Docetaxel was dissolved in the mixture of step 2.

4. Solution of step 3 was filtered through a 0.22 μm filter membrane and filled into vials.

Dilution for one-component compositions of docetaxel:

2 mL of the docetaxel composition was withdrawn using a syringe and injected into 250 mL of 0.9% sodium chloride solution. This mixture was mixed by gentle shaking.

Physical properties of one-component compositions of docetaxel and their diluted formulations:

| | Composition | | |
|---|---|---|---|
| | Comparative | Comparative | Comparative |
| Parameter | Example A | Example B | Example C |
| Syringeability* | Grade 4 | Grade 4 | Grade 4 |
| pH of diluted | | | |
| formulation | | | |
| Initial | ND** | 3.83 | 3.98 |
| After about 3 hours | ND** | 3.94 | 3.98 |
| After 24 hours | 3.92 | 3.81 | 3.93 |
| Visually observed | Precipitation | Precipitation | Precipitation |
| precipitation of diluted | started after 4 | started after 3 | started after 2 |
| formulation | hours | hours, 30 | hours, 45 |
| | | minutes | minutes |

*Syringeability was evaluated based on the following qualitative grading: 1: very easy to syringe; 2: easy to syringe; 3: average syringeability; 4: moderately difficult to syringe, 5: highly difficult to syringe.
**Not done.

### Example 13

### Two-Component Docetaxel Compositions and Premix

| | Grams | | |
|---|---|---|---|
| Ingredient | Component 1 | Component 2 | Pre-mix* |
| Docetaxel trihydrate | 4 | — | 8 |
| SOLUTOL ® HS-15 | 61.8 | 20.6 | 24.72 |
| Ethanol | 31.96 | 31.96 | 25.56 |
| Water | — | 40 | 24 |
| Citric acid anhydrous | 0.3 | — | 0.075 |

*A 2:6 v/v ratio of component 1 to component 2.

Manufacturing process and method of dilution were similar to those described in Example 11.

### Example 14

### Two-Component Compositions Comprising Docetaxel, Solutol and Cremophor ELP, and their Pre-Mix

| | Grams | | |
|---|---|---|---|
| Ingredient | Component 1 | Component 2 | Pre-mix* |
| Docetaxel trihydrate | 4 | — | 8 |
| SOLUTOL ® HS-15 | 61.8 | — | 12.36 |
| Cremophor ® ELP** | — | 20.6 | 12.36 |
| Ethanol | 31.96 | 31.96 | 25.55 |
| Water | — | 40 | 24 |
| Citric acid anhydrous | 0.3 | — | 0.075 |

*A 2:6 v/v ratio of component 1 to component 2.
**Polyethoxylated castor oil, marketed by BASF Corp, New Jersey USA.

Manufacturing process and method of dilution were similar to those described in Example 11.

### Example 15

### Two-Component Compositions Comprising Docetaxel and Alcohol in One Component, and Solutol in Another Component, and their Pre-Mix

| | Grams | | |
|---|---|---|---|
| Ingredient | Component 1 | Component 2 | Pre-mix* |
| Docetaxel trihydrate | 4 | — | 8 |
| SOLUTOL ® HS-15 | — | 61.8 | 37.08 |
| Ethanol | 79.9 | 15.98 | 25.56 |
| Water | — | 20 | 12 |
| Citric acid anhydrous | 0.10 | — | 0.025 |

*A 2:6 v/v ratio of Component 1 to Component 2.

Manufacturing process and method of dilution were similar to those described in Example 11.

Physical properties of the compositions, and their diluted formulations, are given in the table below.

| Parameter | Example 15 |
|---|---|
| Syringeability of component 2 | 2 |
| Syringeability of pre-mix | 2 |
| Visually observed | No precipitation |
| precipitation of diluted | observed over |
| formulation | 22 hours |

### Example 16

### Two-Component Compositions of Docetaxel, Devoid of Solutol

| | Grams | | |
|---|---|---|---|
| Ingredient | Component 1 | Component 2 | Pre-mix* |
| Docetaxel trihydrate | 4 | — | 1 |
| Cremophor ® ELP | — | 61.8 | 37.08 |
| Ethanol | 79.9 | 15.98 | 25.56 |
| Water | — | 20 | 12 |
| Citric acid | 0.1 | — | 0.025 |
| anhydrous | | | |

*A 2:6 v/v ratio of Component 1 to Component 2.

Manufacturing process and method of dilution are similar to those described in Example 11.

### Example 17

### Two-Component Compositions Comprising Docetaxel Forms X and XI

| | mg/mL | | |
|---|---|---|---|
| Ingredient | Component 1 | Component 2 | Pre-mix* |
| Docetaxel Form X | 40 | — | 10 |
| Docetaxel Form XI | — | 40 | 10 |
| Polysorbate 80 | 618 | 206 | 309 |
| Ethanol | 319.6 | 319.6 | 319.6 |
| Water | — | 400 | 300 |
| Citric acid anhydrous | q.s. to pH 3.55 | — | — |

*A 2:6 v/v ratio of Component 1 to Component 2.

Manufacturing process: similar to that of Example 11 except that Solutol was replaced with polysorbate 80.

1. A process for preparing docetaxel, comprising:
a) reacting a compound of Formula B, 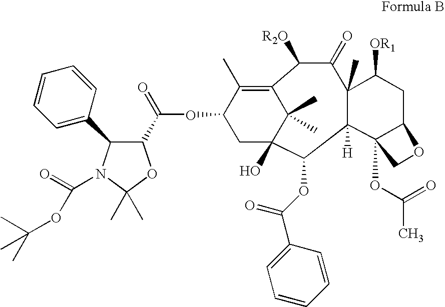
where R₁═R₂═CCl₃—CH₂—OCO—, with zinc and acetic acid in the presence of methanol to give a compound of Formula A, 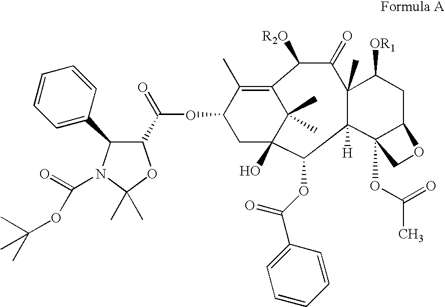
where R₁═R₂═H; and
b) purifying the compound of Formula A using ethyl acetate and n-heptane.

2. A crystalline Form X of docetaxel, characterized by an X-ray powder diffraction pattern having peaks at about 5.3, 8.9, 10.0, 10.6, 11.2, 12.2, 13.7, 14.1, 15.8, 20.4, 21.2, 21.6, and 21.9, ±0.2 degrees two-theta.

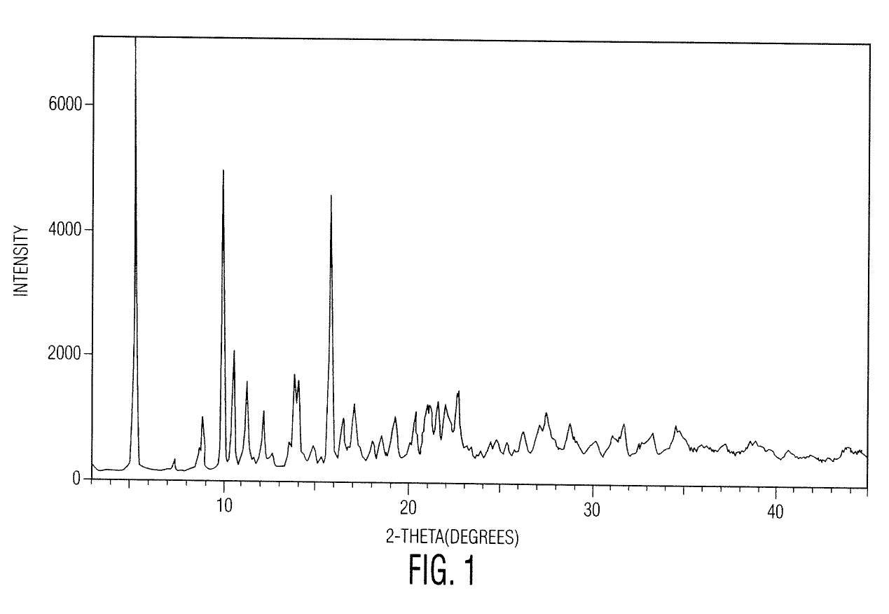

3. The crystalline Form X of docetaxel of claim 2, characterized by having an X-ray powder diffraction pattern substantially as depicted in FIG. 1.

4. The crystalline Form X of docetaxel of claim 2, characterized by having a differential scanning calorimetry thermogram having endothermic peaks at about 106° C. and 175° C.

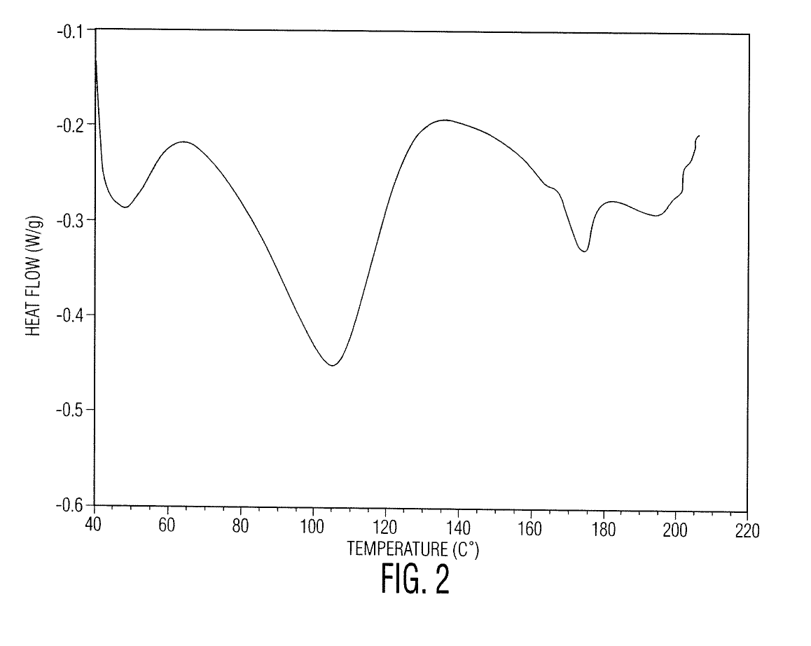

5. The crystalline Form X of docetaxel of claim 2, characterized by having a differential scanning calorimetry thermogram substantially as depicted in FIG. 2.

6. The crystalline Form X of docetaxel of claim 2, characterized by having an infrared absorption spectrum substantially as depicted in FIG. 4.

7. A process for preparing crystalline Form X of docetaxel of claim 2, comprising crystallizing docetaxel from a mixture of ethyl acetate and a hydrocarbon.

8. The process of claim 7, wherein a hydrocarbon comprises n-hexane, n-heptane, or cyclohexane.

9. A crystalline Form XI of docetaxel, characterized by an X-ray powder diffraction pattern having peaks at about 4.4, 4.5, 7.0, 8.0, 8.7, 9.1, 11.0, 11.4, 12.3, 12.5, 13.5, 14.1, 15.4, 16.5, 16.9, 17.4, 18.4, 19.5, and 20.4, ±0.2 degrees two-theta.

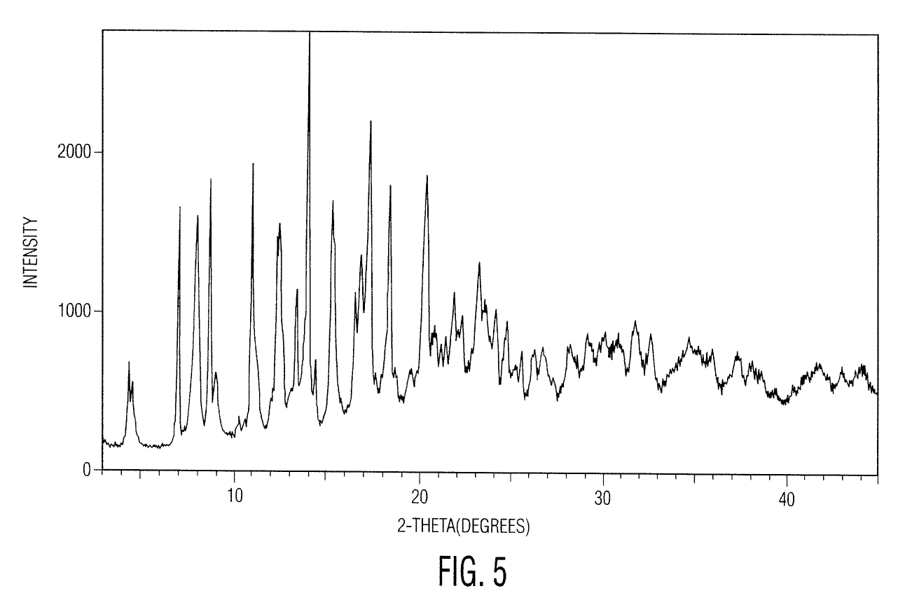

10. The crystalline Form XI of docetaxel of claim 9, characterized by having an X-ray powder diffraction pattern substantially as depicted in FIG. 5.

11. The crystalline Form XI of docetaxel of claim 9, characterized by having a differential scanning calorimetry thermogram having endothermic peaks at about 66° C. and 161° C.

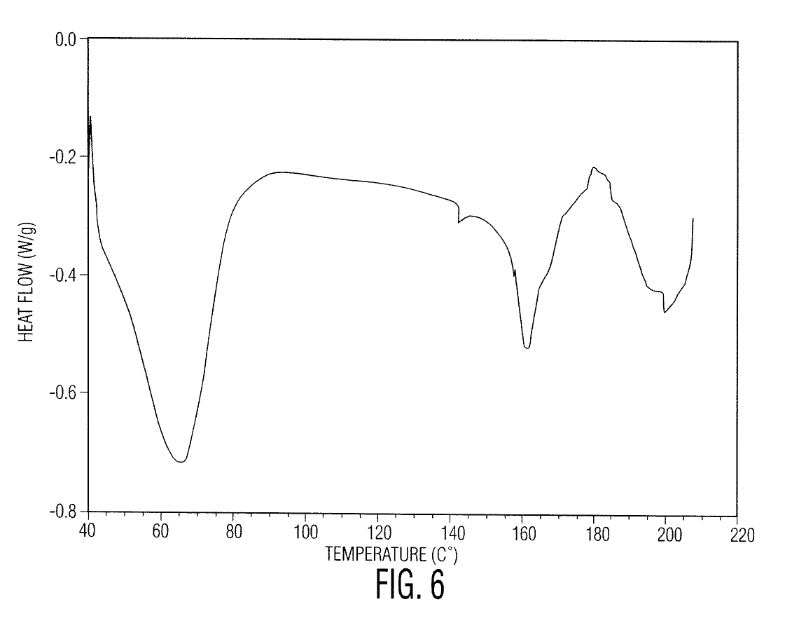

12. The crystalline Form XI of docetaxel of claim 9, characterized by having a differential scanning calorimetry thermogram substantially as depicted in FIG. 6.

13. The crystalline Form XI of docetaxel of claim 9, characterized by having an infrared absorption spectrum substantially as depicted in FIG. 8.

14. A process for preparing crystalline Form XI of docetaxel of claim 9, comprising crystallizing docetaxel from a mixture of acetone and an ether.

15. The process of claim 14 wherein an ether comprises a dialkyl ether.

16. The process of claim 14 wherein an ether comprises diisopropyl ether.

17. A process for preparing docetaxel trihydrate, comprising:
a) combining docetaxel and water;
b) optionally, heating the mixture of step a); and
c) maintaining the mixture at 25-35° C. to effect crystallization.

18. A process for purifying docetaxel, comprising:
a) dissolving docetaxel in an organic solvent;
b) crystallizing docetaxel from the solution by adding an ether; and
c) optionally, recrystallizing the docetaxel obtained.

19. The process of claim 18, wherein an organic solvent comprises acetone, acetonitrile, or a mixture thereof.

20. The process of claim 18, wherein an ether comprises a dialkyl ether.

21. The process of claim 18, wherein an ether comprises diisopropyl ether.

22. A process for purifying docetaxel, comprising:
A. purification using acetonitrile and an ether, comprising:
1) dissolving docetaxel in acetonitrile;
2) crystallizing docetaxel from the solution by adding an ether; and
3) optionally, recrystallizing the docetaxel obtained; then
B. column purification, comprising:
1) dissolving docetaxel in dichloromethane;
2) eluting the docetaxel solution through a silica column using eluents comprising ethyl acetate and n-heptane;
3) recovering eluent fractions containing purified docetaxel;
4) concentrating recovered eluent fractions;
5) crystallizing docetaxel by cooling; then
C. purification using acetone and diisopropyl ether, comprising:
1) dissolving docetaxel in acetone;
2) crystallizing docetaxel from the solution by adding an ether; and
3) optionally, recrystallizing the docetaxel obtained.

23. The process of claim 22, wherein an ether comprises a dialkyl ether.

24. The process of claim 22, wherein an ether comprises diisopropyl ether.

25. Substantially pure docetaxel, prepared by a process of claim 18.

26. The substantially pure docetaxel of claim 25, which is substantially free of isomeric or related impurities.

27. The substantially pure docetaxel of claim 25, which is substantially free of the impurities having Formula II, III, IV, V, VI, VII, VIII, IX, X, XI, XII, XIII, XV, IXX, XX, XIX, XXII, XXIII, XXIV, XXV, and XXVI.

28. The substantially pure docetaxel of claim 27, having less than about 0.15 weight percent of any of the individual impurities.

29. The substantially pure docetaxel of claim 27 having less than about 0.5 weight percent of any combination of the individual impurities.

30. Substantially pure docetaxel, prepared by a process of claim 22.
